(19) United States
(12) Patent Application Publication
(10) Pub. No.: US 2022/0259599 A1
(43) Pub. Date: Aug. 18, 2022

(54) COMPOSITIONS AND METHODS FOR INHIBITING NUCLEIC ACID VECTOR-INDUCED INFLAMMATORY RESPONSES

(71) Applicant: President and Fellows of Harvard College, Cambridge, MA (US)

(72) Inventors: Ying Kai Chan, Cambridge, MA (US); Jessica Chiang, Cambridge, MA (US); George M. Church, Cambridge, MA (US)

(73) Assignee: President and Fellows of Harvard College, Cambridge, MA (US)

(21) Appl. No.: 17/611,062

(22) PCT Filed: May 14, 2020
(86) PCT No.: PCT/US2020/032819

§ 371 Date: Nov. 12, 2021

Related U.S. Application Data
(60) Provisional application No. 62/848,298, filed on May 15, 2019.

(51) Int. Cl. C12N 15/117; C12N 15/86

(52) U.S. Cl. CPC C12N 15/117; C12N 2750/14143; C12N 2310/17; C12N 15/86

(57) ABSTRACT

Provided herein, in some embodiments, are nucleic acid vectors, such as recombinant viral genomes, comprising an inhibitory oligonucleotide that reduces inflammation for use, for example, in gene therapy.

### RELATED APPLICATION

This application claims the benefit under 35 U.S.C. § 119(e) of U.S. provisional application Ser. No. 62/848,298, filed May 15, 2019, which is incorporated by reference herein in its entirety.

### GOVERNMENT LICENSE RIGHTS

This invention was made with government support under HG008525 awarded by National Institutes of Health. The government has certain rights in the invention.

### BACKGROUND

Numerous applications in basic science and the clinic require successful delivery of nucleic acids into a host. For example, viruses have evolved to become highly efficient at nucleic acid delivery to specific cell types while avoiding immunosurveillance by an infected host. These properties make viruses attractive gene-delivery vehicles (vectors) for gene therapy. Several types of viruses, including retrovirus, adenovirus, adeno-associated virus (AAV), and herpes simplex virus, have been modified in the laboratory for use in gene therapy (Robbins, P D et al. Pharmol Ther, 1998;80(1):35-47). Nonetheless, exogenous nucleic acids, including viral vectors have been shown to elicit an inflammatory response.

### SUMMARY

Provided herein, in some embodiments, are variants of a TLR9 inhibitory oligonucleotide TTA4 tested for their ability to inhibit inflammatory responses to DNA when administered in cis fused to the 3′ end of an inflammatory CpG DNA oligonucleotide sequence (referred to herein as ODN 2006). The data provided herein show that all tested TTA4 sequences containing single nucleotide changes retained TLR9 inhibitory activity. Unexpectedly, some variant sequences demonstrate significantly superior inhibition of TLR9-mediated inflammation compared to the control TTA4 sequence. Furthermore, the findings provided herein suggest that two tandem repeats of the TTAGGG telomeric motif administered in cis is the minimal sequence required for TLR9 inhibitory activity. Thus, these novel TLR9 inhibitory sequences, in some embodiments, can be incorporated into a larger piece of inflammatory nucleic acid (e.g., DNA,) such as a viral vector, to effectively inhibit unwanted immune responses.

Provided herein, in some embodiments, are molecular therapy vectors (e.g., viral molecule therapy vectors) that inhibit nucleic acid-mediated inflammatory responses while boosting expression of a desired therapeutic molecule (e.g., a therapeutic gene of interest). The vectors of the present disclosure include a recombinant viral genome linked in cis to an inhibitory oligonucleotide that prevents production (e.g., virally-induced production) of proinflammatory cytokines. In some embodiments, the inhibitory oligonucleotide inhibits nucleic acid-mediated activation of toll-like receptors and/or inhibits nucleic acid-mediated toll-like receptor (TLR) signaling (e.g., TLR9). Surprisingly, inclusion of the inhibitory oligonucleotide in the nucleic acid vector (e.g., viral genome) not only inhibits the inflammatory response, but it also increases transduction efficiency and/or efficacy of the therapeutic nucleotide sequence and/or expression levels of expression products encoded by nucleic acids, as appropriate. Thus, the amount of a nucleic acid vector (e.g., recombinant viral genome) needed to be therapeutically effective, in some embodiments, is less than the amount needed with conventional viral vector delivery systems that do not include an inhibitory oligonucleotide.

Thus, some aspects of the present disclosure provide inflammatory nucleic acids (e.g., recombinant viral genomes) comprising a therapeutic nucleotide sequence (e.g., DNA encoding a gene (e.g., Cas9) or gene fragment (e.g., a replacement exon of interest)) and an inhibitory oligonucleotide that inhibits the production of proinflammatory cytokines. Also provided herein, in some aspects, are methods comprising administering to a subject an inflammatory nucleic acid (e.g., recombinant viral genome) that comprises a therapeutic nucleotide sequence (e.g., DNA) and an inhibitory oligonucleotide that inhibits the production of proinflammatory cytokines (e.g., by inhibiting TLR activation and/or inhibiting TLR signaling). In some embodiments, the inflammatory nucleic acids (e.g., recombinant viral genomes) are administered intramuscularly. In other embodiments, the inflammatory nucleic acids (e.g., recombinant viral genomes) are administered intravenously. In some embodiments, the inflammatory nucleic acids (e.g., recombinant viral genomes) are administered to the eye (e.g., intravitreally and subretinally). In some embodiments, the inflammatory nucleic acids (e.g., recombinant viral genomes) are administered to the central nervous system (e.g., intrathecally and intracranially).

Other aspects of the present disclosure provide inflammatory nucleic acids (e.g., recombinant viral genomes) comprising a therapeutic nucleotide sequence and an inhibitory oligonucleotide that inhibits nucleic acid-sensing TLR activation and/or signaling (e.g., by inhibiting TLR activation and/or inhibiting TLR signaling). Also provided herein, in some aspects, are methods comprising administering to a subject an inflammatory nucleic acid (e.g., recombinant viral genome) comprising a therapeutic nucleotide sequence and an inhibitory oligonucleotide that inhibits nucleic acid-sensing TLR activation and/or signaling.

In some embodiments, the TLR is TLR9. In some embodiments, the inhibitory oligonucleotide binds to the TLR. In some embodiments, the inhibitory oligonucleotide binds to inflammatory nucleic acids.

Still other aspects of the present disclosure provide nucleic acid vectors comprising: (a) an inhibitory oligonucleotide variant comprising an inhibitory nucleotide sequence that comprises a nucleotide substitution at one or more of positions 1, 2, 3, 7, 8, 9, 13, 14, 15, 19, 20, and 21, relative to the nucleotide sequence of SEQ ID NO: 2 (TTAGGGTTAGGGTTAGGGTTAGGG); and (b) a therapeutic nucleotide sequence.

Further aspects of the present disclosure provide nucleic acid vectors comprising: (a) an inhibitory oligonucleotide variant comprising an inhibitory nucleotide sequence that comprises a nucleotide substitution at one or more of positions 4, 5, 6, 10, 11, 12, 16, 17, 18, 22, 23, and 24, relative to the nucleotide sequence of SEQ ID NO: 2 (TTAGGGTTAGGGTTAGGGTTAGGG); and (b) a therapeutic nucleotide sequence.

Yet other aspects of the present disclosure provide nucleic acid vectors comprising: (a) an inhibitory oligonucleotide consisting of SEQ ID NO: 41 or 42; and (b) a therapeutic nucleotide sequence.

Additional aspects of the present disclosure provide methods of treating a subject, comprising administering to the subject any of the nucleic acid vectors disclosed herein.

It should be understood that the terms oligonucleotide, nucleic acid, and nucleotide sequence are used interchangeably herein.

### BRIEF DESCRIPTION OF THE DRAWINGS

FIG. 1 is a schematic representation of a single-stranded DNA oligonucleotide comprising the inflammatory nucleic acid sequence ODN 2006 fused to the TLR9 inhibitory oligonucleotide TTA4 (SEQ ID NO: 2). The positions mutated in the present disclosure are indicated in underlined and bold text, and the numbering used to denote each position is shown above the first nucleotide of each TTAGGG motif.

FIGS. 2A-2E include in vitro data showing the effect of mutating the indicated positions of the TLR9 inhibitory oligonucleotide TTA4 on inflammation induced by the TLR9 activating sequence ODN 2006. Data are shown as mean inflammation±s.d. as a percentage of the ODN 2006-TTA4 control. n=3 technical replicates and data are representative of at least two independent experiments. *P<0.05, **P<0.01, ***P<0.001 (unpaired t-test); n.s., not statistically significant. FIG. 2A shows that ODN 2006 fused to the TTA4 variants at positions 1-3 indicated on the x-axis were tested using the HEK293-TLR9 reporter cell assay. FIG. 2B shows that ODN 2006 fused to the TTA4 variants at positions 7-9 indicated on the x-axis were tested using the HEK293-TLR9 reporter cell assay. FIG. 2C shows that ODN 2006 fused to the TTA4 variants at positions 13-15 indicated on the x-axis were tested using the HEK293-TLR9 reporter cell assay. FIG. 2D shows that ODN 2006 fused to the TTA4 variants at positions 19-21 indicated on the x-axis were tested using the HEK293-TLR9 reporter cell assay. FIG. 2E shows that ODN 2006 fused to the TTA4 variants indicated on the x-axis were tested using the HEK294-TLR9 reporter cell assay.

FIG. 3 is a schematic representation of a single-stranded DNA oligonucleotide comprising the inflammatory nucleic acid sequence ‘ODN 2006’ fused to four copies of the TTAGGG motif (SEQ ID NO: 2). The constructs tested in the present disclosure contained one (TTA1), two (TTA2), three (TTA3), or four (TTA4) copies of the TTAGGG motif.

FIG. 4 includes in vitro data showing the effect of encoding the indicated number of copies of the TTAGGG motif on inflammation induced by the TLR9 activating sequence ODN 2006. Data are shown as mean inflammation±s.d. as a percentage of the indicated ODN 2006-TTA control sequence. n=3 technical replicates and data are representative of three independent experiments. *P<0.05, **P<0.01 (unpaired t-test); n.s., not statistically significant.

FIG. 5 is a schematic representation of a single-stranded DNA oligonucleotide comprising the inflammatory nucleic acid sequence ODN 2006 fused to the TLR9 inhibitory oligonucleotide TTA4 (SEQ ID NO: 2). The positions mutated are indicated in underlined and bold text, and the numbering used to denote each position is shown above the first nucleotide of each GGG motif.

FIGS. 6A-6D include in vitro data showing the effect of mutating the indicated positions of the TLR9 inhibitory oligonucleotide TTA4 on inflammation induced by the TLR9 activating sequence ODN 2006. HEK293-TLR9 reporter cells were incubated with ODN 2006 fused to each of the TTA4 variant sequences indicated on the x-axis (single-stranded DNA with a phosphorothioate backbone). Data are shown as mean inflammation±s.d. as a percentage of the ODN 2006-TTA4 control. n=3 technical replicates and data are representative of at least two independent experiments. Asterisks represent p-values for each TTA4 variant sequence relative to TTA4WT; *P<0.05, **P<0.01, ***P<0.001 (unpaired t-test). FIG. 6A shows ODN 2006 fused to the TTA4 variants at positions 4-6 indicated on the x-axis were tested using the HEK293-TLR9 reporter cell assay. FIG. 6B shows ODN 2006 fused to the TTA4 variants at positions 10-12 indicated on the x-axis were tested using the HEK293-TLR9 reporter cell assay. FIG. 6C shows ODN 2006 fused to the TTA4 variants at positions 16-18 indicated on the x-axis were tested using the HEK293-TLR9 reporter cell assay. FIG. 6D shows ODN 2006 fused to the TTA4 variants at positions 22-24 indicated on the x-axis were tested using the HEK293-TLR9 reporter cell assay.

### DETAILED DESCRIPTION

Despite recent advances, the effectiveness of nucleic acid vectors (e.g., viral nucleic vectors) as gene delivery vehicles has been limited, in part, due to vector-induced inflammation. Thus, clinically, gene therapy with nucleic acid vectors (e.g., viral nucleic acid vectors) often includes systemic treatment with an immunosuppressive agent. Such immunosuppressive and anti-inflammatory drugs, however, can compromise the patient's immune system during treatment, and patients often still develop neutralizing antibodies or T cells to exogenous biological materials (e.g., against AAV capsid), precluding additional administration or leading to destruction of transduced cells.

Provided herein, in some embodiments, are nucleic acid vectors (e.g., recombinant viral genomes) that may be used as delivery vehicles, without inducing a substantial inflammatory response. In some embodiments, the nucleic acid vectors (e.g., recombinant viral genomes) induce less of an inflammatory response than would otherwise be induced without the presence of the inhibitory oligonucleotide. The vectors, e.g., recombinant viral genomes, of the present disclosure include an inhibitory oligonucleotide that inhibits production of proinflammatory cytokines, thus, inhibiting the inflammatory response. In some embodiments, the inhibitory oligonucleotide inhibits an inflammatory response induced by the toll-like receptor (TLR) pathway, for example, by inhibiting (preventing) TLR activation and/or inhibiting TLR signaling.

### Inflammatory Response to Nucleic Acid Vectors

Nucleic acid vectors (e.g., viral vectors) are often studied in experimental and clinical models as agents for gene therapy. Recent generations of viral vectors, for example, have the majority of viral genes removed and result in vectors with a large carrying capacity, reduced host immune responses, and improved gene transfer efficiency. Some viral vectors, such as adenovirus vectors and adeno-associated virus vectors, however, still activate innate immune responses following administration in vivo. Unlike the adaptive response, the innate response to viral vectors is independent of immunological memory and is caused by recognition of conserved features of microbes, commonly termed pattern-associated molecular patterns (PAMPs). This response results in inflammation of transduced tissues and can reduce viral transduction efficiency. Viral infection can activate a number of signaling pathways following cell entry that ultimately lead to expression of inflammatory (proinflammatory) genes. Various cytokines, chemokines and leukocyte adhesion molecules are induced by the viral vector in a wide range of cell types providing a molecular basis for the inflammatory properties of these vectors. See Liu, Q. et al. Gene Therapy 2003;10:935-940. In some embodiments, administration of a viral genome comprising an inhibitory nucleotide sequence as provided herein reduces NK-κB activation in a subject by at least 40%, at least 50%, at least 60%, at least 70%, at least 80, at least 90%, or at least 98%, relative to a control. In some embodiments, the control is NK-κB activation in the subject administered a nucleic acid vector (e.g., a recombinant viral genome) without an inhibitory nucleotide sequence.

### Toll-like Receptor Signaling Pathway

One of the signaling pathways activated following in vivo administration (in a subject) of viral vectors is the toll-like receptor (TLR) signaling pathway. TLRs are immune pattern-recognition receptors that detect pathogens and damaged cells. For example, TLR9 is well-known, and TLR9 amino acid sequences can be found in publically-available gene databases, such as GenBank and UniProtKB. For example, the amino acid sequence of wild-type human TLR9 can be identified as UniProtKB entryQ9NR96 (TLR9_Human).

TLR9 is generally located on endosomal membranes in immune cells. TLR9 is an exemplary nucleic acid-sensing TLRs that detects exogenous nucleic acids (inflammatory nucleic acids) that have entered a cell (see, e.g. Takeda, K et al., Semin Immunol. 2004;16(1):3-9; Lee, Jet al. Proc Natl Acad Sci USA. 2011;108(34):14055-60). Nucleic acids recognized by TLR9 include those that originate from bacteria, viruses, or even endogenous nucleic acids. ‘Nucleic acid-sensing TLRs’ are TLRs that can bind to nucleic acids, such as ssRNA, dsRNA, and DNA containing unmethylated CpG (cytosine-phosphate-guanine) motifs. This binding typically results in the dimerization of TLR and activation of TLR signaling, which results in the production of (expression of and/or activation of) proinflammatory molecules, such as proinflammatory cytokines. In particular, TLR9 recognizes nucleic acids with unmethylated CpGs (Kumagai, Y, et al. Adv Drug Deliv Rev. 2008;60(7)795-804).

As used herein, unless otherwise specified, ‘inflammatory nucleic acids’ are nucleic acids that activate TLR signaling (e.g., bind to TLR to activate TLR signaling). In some embodiments, the inflammatory nucleic acids comprise deoxycytidyl-deoxyguanosine (CpG) oligodeoxynucleotides. CpG oligodeoxynucleotides are sequences that comprise at least one unmethylated CpG motif and activate an immune response. See, e.g., Krieg, A M et al. Nature.

1995; 374(6522):546-9. In some embodiments, the inflammatory nucleic acids (e.g., CpG oligodeoxynucleotides) activate TLR9 signaling (e.g., by binding to TLR9). An exemplary inflammatory nucleic acid is provided in Example 1 (ODN 2006).

TLR signaling results in an inflammatory response characterized by gene expression of antiviral molecules and proinflammatory cytokines, including type I interferons and NF-κB (p25-RelA complex) target genes. Thus, TLR signaling may be used as means to determine the impact of an inhibitory oligonucleotide on the inflammatory response. In some embodiments, a TLR reporter cell line may be used to assess the inhibitory nature of an oligonucleotide, for example, by determining the level of cytokine (e.g. IL6, CXCL10 and/or TNF) production, which reflects the level of TLR signaling. As an example, recognition of unmethylated CpG motifs in exogenous DNA activates TLR9, and TLR9 signaling leads to an increase in expression of proinflammatory cytokines, including IL6, CXCL10, and/or TNF. See, e.g., Krieg A M Nat Rev Drug Discov. 2006;5(6):471-84. In some embodiments, the level of a particular cytokine is measured using quantitative PCR with primers targeting the cytokine of interest. Additional methods of measuring cytokine levels include enzyme-linked immunosorbent assay

(ELISA) and Western blot analysis with an anti-cytokine antibody.

Inhibition of an inflammatory response may be measured as a decrease in TLR signaling. For example, a decrease in cytokine activity level or expression level (e.g., a 2-fold, 5-fold, 10-fold, 50-fold reduction) relative to a control may indicate inhibition (partial or complete inhibition) of the inflammatory response.

Inhibitory Oligonucleotides

An inhibitory oligonucleotide is an oligonucleotide that, when co-delivered in vivo with another nucleic acid (such as a viral genome, a single-stranded RNA, or a single-stranded DNA), inhibits the production of proinflammatory cytokines, relative to proinflammatory cytokine production in the absence of the inhibitory oligonucleotide. See, e.g., Stunz, L L et al. Eur J Immunol. 2002;32(5):1212-22; Lenert, Pet al. DNA Cell Biol. 2003;22(10):621-31; Lenert, Pet al. Arthritis Res Ther. 2009;11(3):R79; Lenert, P S et al. Arthritis Res Ther. 2006;8(1):203; Kaminski, J J et al. J Immunol. 2013;191(7):3876-83; Shirota, H et al. J Immunol. 2005;174(8):4579-83; Peter, Met al. Immunology. 2008;123(1):118-28. An inhibitory oligonucleotide may inhibit the production of proinflammatory cytokines, for example, by at least 50%, at least 60%, at least 70%, at least 80%, at least 85%, at least 90%, at least 95%, or at least 98% relative to proinflammatory cytokine production in the absence of the inhibitory oligonucleotide. Inhibitory nucleotides typically inhibit the production of proinflammatory cytokines by inhibiting TLR activation and/or inhibiting TLR signaling. The inhibitory oligonucleotides of the present disclosure comprise at least two nucleotides covalently linked together, and in some instances, may contain phosphodiester bonds (e.g., a phosphodiester “backbone”). In some instances, the inhibitory oligonucleotide may contain phosphorothioate bonds (e.g., a phosphorothioate backbone). The length of an inhibitory oligonucleotide may vary. In some embodiments, the length of an inhibitory oligonucleotide is 4 to 200 nucleotides. In some embodiments, an inhibitory oligonucleotide has a length of 4 to 100 nucleotides. In some embodiments, an inhibitory oligonucleotide has a length of 4 to 10, 4 to 20, 4 to 30, 4 to 50, 4 to 60, 4 to 70, 4 to 80, or 4 to 90 nucleotides. In some embodiments, an inhibitory oligonucleotide has a length of 5 to 10, 5 to 20, 5 to 30, 5 to 50, 5 to 60, 5 to 70, 5 to 80, 5 to 90, or 5 to 100 nucleotides. In some embodiments, an inhibitory oligonucleotide has a length of 6 to 10, 6 to 20, 6 to 30, 6 to 50, 6 to 60, 6 to 70, 6 to 80, 6 to 90, or 6 to 100 nucleotides. In some embodiments, an inhibitory oligonucleotide has a length of 7 to 10, 7 to 20, 7 to 30, 7 to 50, 7 to 60, 7 to 70, 7 to 80, 7 to 90, or 7 to 100 nucleotides. In some embodiments, an inhibitory oligonucleotide has a length of 8 to 10, 8 to 20, 8 to 30, 8 to 50, 8 to 60, 8 to 70, 8 to 80, 8 to 90, or 8 to 100 nucleotides. In some embodiments, an inhibitory oligonucleotide has a length of 9 to 10, 9 to 20, 9 to 30, 9 to 50, 9 to 60, 9 to 70, 9 to 80, 9 to 90, or 9 to 100 nucleotides. In some embodiments, an inhibitory oligonucleotide has a length of 10 to 10, 10 to 20, 10 to 30, 10 to 50, 10 to 60, 10 to 70, 10 to 80, 10 to 90, or 10 to 100 nucleotides. Inhibitory oligonucleotides may be produced recombinantly or synthetically, for example. In some embodiments, the inhibitory oligonucleotide comprises (or consists of or consists essentially of) deoxyribonucleotides. Thus, in some embodiments, the inhibitory oligonucleotide is an inhibitory DNA oligonucleotide. In some embodiments, the inhibitory oligonucleotide does not include RNA. It should be understood that the definition of inhibitory oligonucleotides, as provided herein, specifically excludes RNA interference molecules (RNAi), such as short interfering RNA (siRNA) molecules.

In some embodiments, the inhibitory oligonucleotides inhibit the activation of nucleic acid-sensing TLRs. For example, the inhibitory oligonucleotides may act as molecular scavengers and bind to (and sequester) inflammatory nucleic acids, thus preventing the inflammatory nucleic acids from binding the TLR and activating TLR signaling. In some embodiments, the inhibitory oligonucleotides prevent dimerization of a TLR.

In some embodiments, the inhibitory oligonucleotides inhibit TLR signaling (to downstream molecules). For example, the inhibitory oligonucleotides may bind indirectly or directly to a TLR (e.g., TLR9) to block TLR-mediated production of proinflammatory cytokines (e.g., induction of proinflammatory cytokine activity and/or expression). See, e.g. Lenert, P S Mediators Inflamm. 2010;2010:986596; Ohto, U et al. Nature. 2015;520(7549):702-5; Lee, J et al. Proc Natl Acad Sci U S A. 2011;108(34):14055-60. In some embodiments, the inhibitor oligonucleotide competes for receptor-mediated endocytosis or phagocytosis. In some embodiments, the inhibitor oligonucleotide inhibits TLR9 trafficking. In some embodiments, the inhibitor oligonucleotide inhibits TLR9 processing into a functionally active product. In some embodiments, the inhibitor oligonucleotide inhibits endosomal acidification or activity of key proteases in endosomes. In some embodiments, the inhibitor oligonucleotide blocks signaling proteins downstream of TLR9.

It should be understood that the term “inhibits” encompasses complete (100%) inhibition and partial (less than 100%) inhibition, otherwise referred to as reduction. Thus, an inhibitory oligonucleotide may reduce nucleic acid-sensing TLR activation and/or signaling by at least 10%, at least 20%, at least 30%, at least 40%, at least 50%, at least 60%, at least 70%, at least 80%, at least 90%, or at least 95%, relative to control (nucleic acid-sensing TLR activation and/or signaling in the absence of the inhibitory oligonucleotide). Complete inhibition refers to a non-detectable, for example, non-measurable amount of activity. That is, a particular activity is considered herein to be completely inhibited if the activity cannot be detected by an assay used by one skilled in the art to detect that particular activity.

In some embodiments, the inhibitory oligonucleotides of the present disclosure inhibit production of proinflammatory cytokines. Non-limiting examples of proinflammatory cytokines include interleukins (e.g., IL-1, IL-6, IL-17 and IL-18), interferons (IFNs, e.g., interferon α (IFNα), interferon β (IFNβ), and interferon γ (IFNγ)), tumor necrosis factors (TNFs) (e.g., TNF-α) and chemokines (e.g., CCL2, CXCL10 and CCLS). In some embodiments, the inhibitory oligonucleotide inhibits IL-6, CXCL10 and/or TNF production. In some embodiments, the inhibitory oligonucleotide inhibits IL-6 production. In some embodiments, the inhibitory oligonucleotide inhibits CXCL10 production. In some embodiments, the inhibitory oligonucleotide inhibits TNF production. As discussed herein, the level of inflammatory cytokine production may be measured using Western blot analysis, quantitative PCR and/or enzyme-linked immunosorbent assay. Other assays for assessing the inflammatory response may be used.

In some embodiments, an inhibitory oligonucleotide reduces production of (activity of and/or expression of) proinflammatory cytokines by at least 10%, at least 20%, at least 30%, at least 40%, at least 50%, at least 60%, at least 70%, at least 80%, at least 90%, or at least 95%, relative to control (inflammatory cytokine production in the absence of the inhibitory oligonucleotide). In some embodiments, an inhibitory oligonucleotide reduces production of (activity of and/or expression of) proinflammatory cytokines by 50%-100%, 60%-100%, 70%-100%, 80%-100%, or 90%-100%, relative to control (inflammatory cytokine production in the absence of the inhibitory oligonucleotide).

In some embodiments, an inhibitory oligonucleotide comprises at least one TTAGGG motif. For example, an inhibitory oligonucleotide may comprise at least 2, 3, 4, 5, 6, 7, 8, 9 or 10 TTAGGG motifs. In some embodiments, an inhibitory oligonucleotide comprises 2 to 5, 2 to 10, or 5 to 10 TTAGGG motifs. In some embodiments, the inhibitory oligonucleotide includes two TTAGGG motifs. In some embodiments, the inhibitory oligonucleotide includes three TTAGGG motifs. In some embodiments, the inhibitory oligonucleotide includes four TTAGGG motifs.

In some embodiments, an inhibitory oligonucleotide consists of 1, 2, 3, 4, 5, 6, 7, 8, 9 or 10 TTAGGG motifs. In some embodiments, an inhibitory oligonucleotide consists of 2 to 5, 2 to 10, or 5 to 10 TTAGGG motifs. In some embodiments, the inhibitory oligonucleotide consists of two TTAGGG motifs. In some embodiments, the inhibitory oligonucleotide consists of three TTAGGG motifs. In some embodiments, the inhibitory oligonucleotide consists of four TTAGGG motifs.

In some embodiments, an inhibitory oligonucleotide comprises at least one nucleotide sequence of any one of SEQ ID NOS: 4-40. In some embodiments, an inhibitory oligonucleotide comprises at least one nucleotide sequence of any one of 47-82. In some embodiments, an inhibitory oligonucleotide consists of the nucleotide sequence of SEQ ID NO: 41. In some embodiments, an inhibitory oligonucleotide consists of the nucleotide sequence of SEQ ID NO: 42. In some embodiments, the inhibitory oligonucleotide includes multiple tandem repeats of a nucleotide sequence of any one of SEQ ID NOS: 4-40. In some embodiments, the inhibitory oligonucleotide includes multiple tandem repeats of a nucleotide sequence of any one of SEQ ID NOS: 47-82. As used herein, unless otherwise noted, tandem repeats are sequences that follow one another. In some examples, the tandem repeats may be directly next to one another (e.g., TTAGGGTTAGGGTTAGGG (SEQ ID NO:41) (repeated sequence underlined)). In some examples, the tandem repeats may be separated by another sequence (e.g., a linker sequence) (e.g., TTAGGG-linker-TTAGGG-linker-TTAGGG). In some embodiments, the inhibitory oligonucleotide includes multiple tandem repeat sequences (e.g. two, three, four or five tandem repeats).

In some embodiments, the multiple tandem repeat sequences in an inhibitory oligonucleotide are separated by a linker. The linker may be oriented in the sense or antisense direction. As an example, a linker may be oriented in the sense direction if it is separating multiple tandem repeat sequences that are also oriented in the sense direction. In some embodiments, a linker is oriented in the antisense direction when it is separating multiple tandem repeat sequences that are also oriented in the antisense direction.

In some embodiments, the linker is a polyA linker (a string of “A” nucleotides).

In some embodiments, the inhibitory oligonucleotide includes a combination of nucleotide sequences of any one of SEQ ID NOS: 4-40. In some embodiments, the inhibitory oligonucleotide includes a combination of nucleotide sequences of any one of SEQ ID NOS: 47-82. For example, the inhibitory oligonucleotide may include at least one copy (e.g., one, two, three, four, five, six or seven copies) of a nucleotide sequence of any one of SEQ ID NOS: 4-40 combined with at least one copy (e.g., one, two, three, four, five, six or seven copies) of a nucleotide sequence of any one of SEQ ID NOS: 4-40. As another example, the inhibitory oligonucleotide may include at least one copy (e.g., one, two, three, four, five, six or seven copies) of a nucleotide sequence of any one of SEQ ID NOS: 47-82 combined with at least one copy (e.g., one, two, three, four, five, six or seven copies) of a nucleotide sequence of any one of SEQ ID NOS: 47-82.

A single expression construct (e.g., ssAAV) may include more than one (e.g., at least 2, at least 3, at least 4, at least 5, at least 6, at least 7, at least 8, at least 9, at least 10, at least 15, at least 20, 2 to 20, 5 to 20, 10 to 20, or 15 to 20) type of inhibitory oligonucleotides, one type (one or more copies, e.g., 2, 3, 4 or 5 tandem copies) in the sense orientation and another type (one or more copies, e.g., 2, 3, 4 or 5 tandem copies) in the antisense orientation. Without being bound by a particular theory, inclusion of two different types of inhibitory oligonucleotides may prevent self-annealing between the inhibitory oligonucleotides and formation of unwanted hairpin structures. In some embodiments, a recombinant single-stranded viral genome comprising an inhibitory oligonucleotide in the sense orientation and another inhibitory oligonucleotide in the antisense direction increases the probability that each packaged viral genome comprises at least one inhibitory oligonucleotide in the correct orientation.

It should be understood that the present disclosure encompasses the use of any inhibitory oligonucleotide, such as those described herein, as well as inhibitory oligonucleotides that share a certain degree of sequence identity (percent identity) with a reference inhibitory oligonucleotide. Percent identity refers to a relationship between the sequences of two or more polynucleotides (nucleic acids), as determined by comparing the sequences. Identity measures the percent of identical matches between the smaller of two or more sequences with gap alignments (if any) addressed by a particular mathematical model or computer program (e.g., “algorithms”). Identity of related molecules can be readily calculated. “Percent (%) identity” as it applies to nucleic acid sequences is defined as the percentage of nucleic acid residues in the candidate nucleic acid sequence that are identical with the residues in the nucleic acid sequence of a second sequence after aligning the sequences and introducing gaps, if necessary, to achieve the maximum percent identity. Identity depends on a calculation of percent identity but may differ in value due to gaps and penalties introduced in the calculation. Variants of a particular inhibitory oligonucleotide may have at least 90%, 91%, 92%, 93%, 94%, 95%, 96%, 97%, 98%, 99% but less than 100% sequence identity to that particular reference inhibitory oligonucleotide, as determined by sequence alignment programs and parameters.

Thus, in some embodiments, an inhibitory oligonucleotide comprises a (at least one) nucleotide sequence having at least 80% identity to the nucleotide sequence of any one of SEQ ID NOS: 4-40. In some embodiments, an inhibitory oligonucleotide comprises a nucleotide sequence having at least 90% identity to the nucleotide sequence of any one of SEQ ID NOS: 4-40. In some embodiments, an inhibitory oligonucleotide comprises a nucleotide sequence having at least 95% identity to the nucleotide sequence of any one of SEQ ID NOS: 4-40. In some embodiments, an inhibitory oligonucleotide comprises a nucleotide sequence having no more than 2 amino acid substitutions, relative to the nucleotide sequence of any one of SEQ ID NOS: 4-40. In some embodiments, an inhibitory oligonucleotide comprises a nucleotide sequence having no more than 1 nucleotide substitution, relative to the nucleotide sequence of any one of SEQ ID NOS: 4-40.

In some embodiments, an inhibitory oligonucleotide comprises a (at least one) nucleotide sequence having at least 80% identity to the nucleotide sequence of any one of SEQ ID NOS: 47-82. In some embodiments, an inhibitory oligonucleotide comprises a nucleotide sequence having at least 90% identity to the nucleotide sequence of any one of SEQ ID NOS: 47-82. In some embodiments, an inhibitory oligonucleotide comprises a nucleotide sequence having at least 95% identity to the nucleotide sequence of any one of SEQ ID NOS: 47-82. In some embodiments, an inhibitory oligonucleotide comprises a nucleotide sequence having no more than 2 amino acid substitutions, relative to the nucleotide sequence of any one of SEQ ID NOS: 47-82. In some embodiments, an inhibitory oligonucleotide comprises a nucleotide sequence having no more than 1 nucleotide substitution, relative to the nucleotide sequence of any one of SEQ ID NOS: 47-82.

The comparison of sequences and determination of percent identity between two sequences can be accomplished using a mathematical algorithm. Techniques for determining identity are codified in publicly available computer programs. Exemplary computer software to determine homology between two sequences include, but are not limited to, the GCG program package (Devereux, J. et al. Nucleic Acids Research, 12(1): 387, 1984), the BLAST suite (Altschul, S. F. et al. Nucleic Acids Res. 25: 3389, 1997), and FASTA (Altschul, S. F. et al. J. Molec. Biol. 215: 403, 1990). Other techniques include: the Smith-Waterman algorithm (Smith, T. F. et al. J. Mol. Biol. 147: 195, 1981; the Needleman-Wunsch algorithm (Needleman, S. B. et al. J. Mol. Biol. 48: 443, 1970; and the Fast Optimal Global Sequence Alignment Algorithm (FOGSAA) (Chakraborty, A. et al. Sci Rep.3: 1746, 2013).

In some embodiments, a nucleic acid vector comprises an inhibitory oligonucleotide variant comprising an inhibitory nucleotide sequence that comprises a nucleotide substitution at one or more (at least one) of positions 1, 2, 3, 7, 8, 9, 13, 14, 15, 19, 20, and 21, relative to the nucleotide sequence of SEQ ID NO: 2. In some embodiments, the nuclei acid vector further comprises a therapeutic nucleotide sequence (e.g., a therapeutic DNA, a therapeutic RNA, or a nucleotide sequence encoding a therapeutic protein).

In some embodiments, a nucleic acid vector comprises an inhibitory oligonucleotide variant comprising an inhibitory nucleotide sequence that comprises a nucleotide substitution at position 1, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 2, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 3, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 7, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 8, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 9, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 13, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 14, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 15, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 19, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 20, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 21, relative to the nucleotide sequence of SEQ ID NO: 2, and a therapeutic nucleotide sequence.

In some embodiments, a nucleic acid vector comprises an inhibitory oligonucleotide variant comprising an inhibitory nucleotide sequence that comprises a nucleotide substitution at one or more (at least one) of positions 4, 5, 6, 10, 11, 12, 16, 17, 18, 22, 23, and 24, relative to the nucleotide sequence of SEQ ID NO: 2. In some embodiments, the nuclei acid vector further comprises a therapeutic nucleotide sequence (e.g., a therapeutic DNA, a therapeutic RNA, or a nucleotide sequence encoding a therapeutic protein).

In some embodiments, a nucleic acid vector comprises an inhibitory oligonucleotide variant comprising an inhibitory nucleotide sequence that comprises a nucleotide substitution at position 4, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 5, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 6, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 10, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 11, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 12, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 16, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 17, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 18, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 22, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 23, relative to the nucleotide sequence of SEQ ID NO: 2, and a therapeutic nucleotide sequence. In some embodiments, a nucleic acid vector comprises an inhibitory oligonucleotide variant comprising an inhibitory nucleotide sequence that comprises a nucleotide substitution at position 24, relative to the nucleotide sequence of SEQ ID NO: 2, and a therapeutic nucleotide sequence.

In some embodiments, a nucleic acid vector (e.g., a recombinant viral genome) comprises an inhibitory oligonucleotide comprising a nucleotide sequence having at least 95% identity to the nucleotide sequence of any one of SEQ ID NOS: 4-40. In some embodiments, a nucleic acid vector (e.g., a recombinant viral genome) comprises an inhibitory oligonucleotide comprising the nucleotide sequence of any one of SEQ ID NOS: 4-40.

In some embodiments, a nucleic acid vector (e.g., a recombinant viral genome) comprises an inhibitory oligonucleotide comprising a nucleotide sequence having at least 95% identity to the nucleotide sequence of SEQ ID NO: 4. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 4. In some embodiments, a nucleic acid vector (e.g., a recombinant viral genome) comprises an inhibitory oligonucleotide comprising the nucleotide sequence of SEQ ID NO: 4 (or an inhibitory oligonucleotide consisting of or consisting essentially of the nucleotide sequence of SEQ ID NO: 4). In some embodiments, a nucleic acid vector (e.g., a recombinant viral genome) comprises an inhibitory oligonucleotide comprising the nucleotide sequence of SEQ ID NO: 4 and a therapeutic nucleotide sequence (e.g., a therapeutic DNA, a therapeutic RNA, or a nucleotide sequence encoding a therapeutic protein). In some embodiments, a nucleic acid vector (e.g., a recombinant viral genome) comprises an inhibitory oligonucleotide comprising a nucleotide sequence having at least 95% identity to the nucleotide sequence of SEQ ID NO: 5. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 5. In some embodiments, a nucleic acid vector (e.g., a recombinant viral genome) comprises an inhibitory oligonucleotide comprising the nucleotide sequence of SEQ ID NO: 5 (or an inhibitory oligonucleotide consisting of or consisting essentially of the nucleotide sequence of SEQ ID NO: 5). In some embodiments, a nucleic acid vector (e.g., a recombinant viral genome) comprises an inhibitory oligonucleotide comprising the nucleotide sequence of SEQ ID NO: 5 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 6. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 6. In some embodiments, a nucleic acid vector (e.g., a recombinant viral genome) comprises an inhibitory oligonucleotide comprising the nucleotide sequence of SEQ ID NO: 6 (or an inhibitory oligonucleotide consisting of or consisting essentially of the nucleotide sequence of SEQ ID NO: 6). In some embodiments, a nucleic acid vector (e.g., a recombinant viral genome) comprises an inhibitory oligonucleotide comprising the nucleotide sequence of SEQ ID NO: 6 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 7. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 7. In some embodiments, a nucleic acid vector (e.g., a recombinant viral genome) comprises an inhibitory oligonucleotide comprising the nucleotide sequence of SEQ ID NO: 7 (or an inhibitory oligonucleotide consisting of or consisting essentially of the nucleotide sequence of SEQ ID NO: 7). In some embodiments, a nucleic acid vector (e.g., a recombinant viral genome) comprises an inhibitory oligonucleotide comprising the nucleotide sequence of SEQ ID NO: 7 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 8. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 8. In some embodiments, a nucleic acid vector (e.g., a recombinant viral genome) comprises an inhibitory oligonucleotide comprising the nucleotide sequence of SEQ ID NO: 8 (or an inhibitory oligonucleotide consisting of or consisting essentially of the nucleotide sequence of SEQ ID NO: 8). In some embodiments, a nucleic acid vector (e.g., a recombinant viral genome) comprises an inhibitory oligonucleotide comprising the nucleotide sequence of SEQ ID NO: 8 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 9. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 9. In some embodiments, a nucleic acid vector (e.g., a recombinant viral genome) comprises an inhibitory oligonucleotide comprising the nucleotide sequence of SEQ ID NO: 9 (or an inhibitory oligonucleotide consisting of or consisting essentially of the nucleotide sequence of SEQ ID NO: 9). In some embodiments, a nucleic acid vector (e.g., a recombinant viral genome) comprises an inhibitory oligonucleotide comprising the nucleotide sequence of SEQ ID NO: 9 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 10. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 10. In some embodiments, a nucleic acid vector (e.g., a recombinant viral genome) comprises an inhibitory oligonucleotide comprising the nucleotide sequence of SEQ ID NO: 10 (or an inhibitory oligonucleotide consisting of or consisting essentially of the nucleotide sequence of SEQ ID NO: 10). In some embodiments, a nucleic acid vector (e.g., a recombinant viral genome) comprises an inhibitory oligonucleotide comprising the nucleotide sequence of SEQ ID NO: 10 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 11. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 11. In some embodiments, a nucleic acid vector (e.g., a recombinant viral genome) comprises an inhibitory oligonucleotide comprising the nucleotide sequence of SEQ ID NO: 11 (or an inhibitory oligonucleotide consisting of or consisting essentially of the nucleotide sequence of SEQ ID NO: 11). In some embodiments, a nucleic acid vector (e.g., a recombinant viral genome) comprises an inhibitory oligonucleotide comprising the nucleotide sequence of SEQ ID NO: 11 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 12. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 12. In some embodiments, a nucleic acid vector (e.g., a recombinant viral genome) comprises an inhibitory oligonucleotide comprising the nucleotide sequence of SEQ ID NO: 12 (or an inhibitory oligonucleotide consisting of or consisting essentially of the nucleotide sequence of SEQ ID NO: 12). In some embodiments, a nucleic acid vector (e.g., a recombinant viral genome) comprises an inhibitory oligonucleotide comprising the nucleotide sequence of SEQ ID NO: 12 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 13. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 13. In some embodiments, a nucleic acid vector (e.g., a recombinant viral genome) comprises an inhibitory oligonucleotide comprising the nucleotide sequence of SEQ ID NO: 13 (or an inhibitory oligonucleotide consisting of or consisting essentially of the nucleotide sequence of SEQ ID NO: 13). In some embodiments, a nucleic acid vector (e.g., a recombinant viral genome) comprises an inhibitory oligonucleotide comprising the nucleotide sequence of SEQ ID NO: 13 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 14. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 14. In some embodiments, a nucleic acid vector (e.g., a recombinant viral genome) comprises an inhibitory oligonucleotide comprising the nucleotide sequence of SEQ ID NO: 14 (or an inhibitory oligonucleotide consisting of or consisting essentially of the nucleotide sequence of SEQ ID NO: 14). In some embodiments, a nucleic acid vector (e.g., a recombinant viral genome) comprises an inhibitory oligonucleotide comprising the nucleotide sequence of SEQ ID NO: 14 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 15. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 15. In some embodiments, a nucleic acid vector (e.g., a recombinant viral genome) comprises an inhibitory oligonucleotide comprising the nucleotide sequence of SEQ ID NO: 15 (or an inhibitory oligonucleotide consisting of or consisting essentially of the nucleotide sequence of SEQ ID NO: 15). In some embodiments, a nucleic acid vector (e.g., a recombinant viral genome) comprises an inhibitory oligonucleotide comprising the nucleotide sequence of SEQ ID NO: 15 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 16. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 16. In some embodiments, a nucleic acid vector (e.g., a recombinant viral genome) comprises an inhibitory oligonucleotide comprising the nucleotide sequence of SEQ ID NO: 16 (or an inhibitory oligonucleotide consisting of or consisting essentially of the nucleotide sequence of SEQ ID NO: 16). In some embodiments, a nucleic acid vector (e.g., a recombinant viral genome) comprises an inhibitory oligonucleotide comprising the nucleotide sequence of SEQ ID NO: 16 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 17. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 17. In some embodiments, a nucleic acid vector (e.g., a recombinant viral genome) comprises an inhibitory oligonucleotide comprising the nucleotide sequence of SEQ ID NO: 17 (or an inhibitory oligonucleotide consisting of or consisting essentially of the nucleotide sequence of SEQ ID NO: 17). In some embodiments, a nucleic acid vector (e.g., a recombinant viral genome) comprises an inhibitory oligonucleotide comprising the nucleotide sequence of SEQ ID NO: 17 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 18. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 18. In some embodiments, a nucleic acid vector (e.g., a recombinant viral genome) comprises an inhibitory oligonucleotide comprising the nucleotide sequence of SEQ ID NO: 18 (or an inhibitory oligonucleotide consisting of or consisting essentially of the nucleotide sequence of SEQ ID NO: 18). In some embodiments, a nucleic acid vector (e.g., a recombinant viral genome) comprises an inhibitory oligonucleotide comprising the nucleotide sequence of SEQ ID NO: 18 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 19. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 19. In some embodiments, a nucleic acid vector (e.g., a recombinant viral genome) comprises an inhibitory oligonucleotide comprising the nucleotide sequence of SEQ ID NO: 19 (or an inhibitory oligonucleotide consisting of or consisting essentially of the nucleotide sequence of SEQ ID NO: 19). In some embodiments, a nucleic acid vector (e.g., a recombinant viral genome) comprises an inhibitory oligonucleotide comprising the nucleotide sequence of SEQ ID NO: 19 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 20. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 20. In some embodiments, a nucleic acid vector (e.g., a recombinant viral genome) comprises an inhibitory oligonucleotide comprising the nucleotide sequence of SEQ ID NO: 20 (or an inhibitory oligonucleotide consisting of or consisting essentially of the nucleotide sequence of SEQ ID NO: 20). In some embodiments, a nucleic acid vector (e.g., a recombinant viral genome) comprises an inhibitory oligonucleotide comprising the nucleotide sequence of SEQ ID NO: 20 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 21. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 21. In some embodiments, a nucleic acid vector (e.g., a recombinant viral genome) comprises an inhibitory oligonucleotide comprising the nucleotide sequence of SEQ ID NO: 21 (or an inhibitory oligonucleotide consisting of or consisting essentially of the nucleotide sequence of SEQ ID NO: 21). In some embodiments, a nucleic acid vector (e.g., a recombinant viral genome) comprises an inhibitory oligonucleotide comprising the nucleotide sequence of SEQ ID NO:21 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 22. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 22. In some embodiments, a nucleic acid vector (e.g., a recombinant viral genome) comprises an inhibitory oligonucleotide comprising the nucleotide sequence of SEQ ID NO: 22 (or an inhibitory oligonucleotide consisting of or consisting essentially of the nucleotide sequence of SEQ ID NO: 22). In some embodiments, a nucleic acid vector (e.g., a recombinant viral genome) comprises an inhibitory oligonucleotide comprising the nucleotide sequence of SEQ ID NO: 22 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 23. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 23. In some embodiments, a nucleic acid vector (e.g., a recombinant viral genome) comprises an inhibitory oligonucleotide comprising the nucleotide sequence of SEQ ID NO: 23 (or an inhibitory oligonucleotide consisting of or consisting essentially of the nucleotide sequence of SEQ ID NO: 23). In some embodiments, a nucleic acid vector (e.g., a recombinant viral genome) comprises an inhibitory oligonucleotide comprising the nucleotide sequence of SEQ ID NO: 23 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 24. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 24. In some embodiments, a nucleic acid vector (e.g., a recombinant viral genome) comprises an inhibitory oligonucleotide comprising the nucleotide sequence of SEQ ID NO: 24 (or an inhibitory oligonucleotide consisting of or consisting essentially of the nucleotide sequence of SEQ ID NO: 24). In some embodiments, a nucleic acid vector (e.g., a recombinant viral genome) comprises an inhibitory oligonucleotide comprising the nucleotide sequence of SEQ ID NO: 24 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 25. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 25. In some embodiments, a nucleic acid vector (e.g., a recombinant viral genome) comprises an inhibitory oligonucleotide comprising the nucleotide sequence of SEQ ID NO: 25 (or an inhibitory oligonucleotide consisting of or consisting essentially of the nucleotide sequence of SEQ ID NO: 25). In some embodiments, a nucleic acid vector (e.g., a recombinant viral genome) comprises an inhibitory oligonucleotide comprising the nucleotide sequence of SEQ ID NO: 25 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 26. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 26. In some embodiments, a nucleic acid vector (e.g., a recombinant viral genome) comprises an inhibitory oligonucleotide comprising the nucleotide sequence of SEQ ID NO: 26 (or an inhibitory oligonucleotide consisting of or consisting essentially of the nucleotide sequence of SEQ ID NO: 26). In some embodiments, a nucleic acid vector (e.g., a recombinant viral genome) comprises an inhibitory oligonucleotide comprising the nucleotide sequence of SEQ ID NO: 26 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 27. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 27. In some embodiments, a nucleic acid vector (e.g., a recombinant viral genome) comprises an inhibitory oligonucleotide comprising the nucleotide sequence of SEQ ID NO: 27 (or an inhibitory oligonucleotide consisting of or consisting essentially of the nucleotide sequence of SEQ ID NO: 27). In some embodiments, a nucleic acid vector (e.g., a recombinant viral genome) comprises an inhibitory oligonucleotide comprising the nucleotide sequence of SEQ ID NO: 27 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 28. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 28. In some embodiments, a nucleic acid vector (e.g., a recombinant viral genome) comprises an inhibitory oligonucleotide comprising the nucleotide sequence of SEQ ID NO: 28 (or an inhibitory oligonucleotide consisting of or consisting essentially of the nucleotide sequence of SEQ ID NO: 28). In some embodiments, a nucleic acid vector (e.g., a recombinant viral genome) comprises an inhibitory oligonucleotide comprising the nucleotide sequence of SEQ ID NO: 28 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 29. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 29. In some embodiments, a nucleic acid vector (e.g., a recombinant viral genome) comprises an inhibitory oligonucleotide comprising the nucleotide sequence of SEQ ID NO: 29 (or an inhibitory oligonucleotide consisting of or consisting essentially of the nucleotide sequence of SEQ ID NO: 29). In some embodiments, a nucleic acid vector (e.g., a recombinant viral genome) comprises an inhibitory oligonucleotide comprising the nucleotide sequence of SEQ ID NO: 29 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 30. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 30. In some embodiments, a nucleic acid vector (e.g., a recombinant viral genome) comprises an inhibitory oligonucleotide comprising the nucleotide sequence of SEQ ID NO: 30 (or an inhibitory oligonucleotide consisting of or consisting essentially of the nucleotide sequence of SEQ ID NO: 30). In some embodiments, a nucleic acid vector (e.g., a recombinant viral genome) comprises an inhibitory oligonucleotide comprising the nucleotide sequence of SEQ ID NO: 30 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 31. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 31. In some embodiments, a nucleic acid vector (e.g., a recombinant viral genome) comprises an inhibitory oligonucleotide comprising the nucleotide sequence of SEQ ID NO: 31 (or an inhibitory oligonucleotide consisting of or consisting essentially of the nucleotide sequence of SEQ ID NO: 31). In some embodiments, a nucleic acid vector (e.g., a recombinant viral genome) comprises an inhibitory oligonucleotide comprising the nucleotide sequence of SEQ ID NO: 31 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 32. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 32. In some embodiments, a nucleic acid vector (e.g., a recombinant viral genome) comprises an inhibitory oligonucleotide comprising the nucleotide sequence of SEQ ID NO: 32 (or an inhibitory oligonucleotide consisting of or consisting essentially of the nucleotide sequence of SEQ ID NO: 32). In some embodiments, a nucleic acid vector (e.g., a recombinant viral genome) comprises an inhibitory oligonucleotide comprising the nucleotide sequence of SEQ ID NO: 32 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 33. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 33. In some embodiments, a nucleic acid vector (e.g., a recombinant viral genome) comprises an inhibitory oligonucleotide comprising the nucleotide sequence of SEQ ID NO: 33 (or an inhibitory oligonucleotide consisting of or consisting essentially of the nucleotide sequence of SEQ ID NO: 33). In some embodiments, a nucleic acid vector (e.g., a recombinant viral genome) comprises an inhibitory oligonucleotide comprising the nucleotide sequence of SEQ ID NO: 33 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 34. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 34. In some embodiments, a nucleic acid vector (e.g., a recombinant viral genome) comprises an inhibitory oligonucleotide comprising the nucleotide sequence of SEQ ID NO: 34 (or an inhibitory oligonucleotide consisting of or consisting essentially of the nucleotide sequence of SEQ ID NO: 34). In some embodiments, a nucleic acid vector (e.g., a recombinant viral genome) comprises an inhibitory oligonucleotide comprising the nucleotide sequence of SEQ ID NO: 34 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 35. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 35. In some embodiments, a nucleic acid vector (e.g., a recombinant viral genome) comprises an inhibitory oligonucleotide comprising the nucleotide sequence of SEQ ID NO: 35 (or an inhibitory oligonucleotide consisting of or consisting essentially of the nucleotide sequence of SEQ ID NO: 35). In some embodiments, a nucleic acid vector (e.g., a recombinant viral genome) comprises an inhibitory oligonucleotide comprising the nucleotide sequence of SEQ ID NO: 35 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 36. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 36. In some embodiments, a nucleic acid vector (e.g., a recombinant viral genome) comprises an inhibitory oligonucleotide comprising the nucleotide sequence of SEQ ID NO: 36 (or an inhibitory oligonucleotide consisting of or consisting essentially of the nucleotide sequence of SEQ ID NO: 36). In some embodiments, a nucleic acid vector (e.g., a recombinant viral genome) comprises an inhibitory oligonucleotide comprising the nucleotide sequence of SEQ ID NO: 36 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 37. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 37. In some embodiments, a nucleic acid vector (e.g., a recombinant viral genome) comprises an inhibitory oligonucleotide comprising the nucleotide sequence of SEQ ID NO: 37 (or an inhibitory oligonucleotide consisting of or consisting essentially of the nucleotide sequence of SEQ ID NO: 37). In some embodiments, a nucleic acid vector (e.g., a recombinant viral genome) comprises an inhibitory oligonucleotide comprising the nucleotide sequence of SEQ ID NO: 37 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 38. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 38. In some embodiments, a nucleic acid vector (e.g., a recombinant viral genome) comprises an inhibitory oligonucleotide comprising the nucleotide sequence of SEQ ID NO: 38 (or an inhibitory oligonucleotide consisting of or consisting essentially of the nucleotide sequence of SEQ ID NO: 38). In some embodiments, a nucleic acid vector (e.g., a recombinant viral genome) comprises an inhibitory oligonucleotide comprising the nucleotide sequence of SEQ ID NO: 38 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 39. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 39. In some embodiments, a nucleic acid vector (e.g., a recombinant viral genome) comprises an inhibitory oligonucleotide comprising the nucleotide sequence of SEQ ID NO: 39 (or an inhibitory oligonucleotide consisting of or consisting essentially of the nucleotide sequence of SEQ ID NO: 39). In some embodiments, a nucleic acid vector (e.g., a recombinant viral genome) comprises an inhibitory oligonucleotide comprising the nucleotide sequence of SEQ ID NO: 39 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 40. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 40. In some embodiments, a nucleic acid vector (e.g., a recombinant viral genome) comprises an inhibitory oligonucleotide comprising the nucleotide sequence of SEQ ID NO: 40 (or an inhibitory oligonucleotide consisting of or consisting essentially of the nucleotide sequence of SEQ ID NO: 40). In some embodiments, a nucleic acid vector (e.g., a recombinant viral genome) comprises an inhibitory oligonucleotide comprising the nucleotide sequence of SEQ ID NO: 40 and a therapeutic nucleotide sequence (e.g., a therapeutic DNA, a therapeutic RNA, or a nucleotide sequence encoding a therapeutic protein).

In some embodiments, a nucleic acid vector comprises an inhibitory oligonucleotide consisting of SEQ ID NO: 41. In some embodiments, a nucleic acid vector comprises an inhibitory oligonucleotide consisting of SEQ ID NO: 42. In some embodiments, the nucleic acid vector further comprises a therapeutic nucleotide sequence.

In some embodiments, a nucleic acid vector (e.g., a recombinant viral genome) comprises an inhibitory oligonucleotide comprising a nucleotide sequence having at least 95% identity to the nucleotide sequence of SEQ ID NO: 21 and an inhibitory oligonucleotide comprising a nucleotide sequence having at least 95% identity to the nucleotide sequence of SEQ ID NO: 30. In some embodiments, the nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 21 and an inhibitory oligonucleotide comprising a nucleotide sequence having no more than 1 nucleotide substitution, relative to the nucleotide sequence of SEQ ID NO: 30. In some embodiments, a nucleic acid vector (e.g., a recombinant viral genome) comprises an inhibitory oligonucleotide comprising the nucleotide sequence of SEQ ID NO: 21 and an inhibitory oligonucleotide comprising the nucleotide sequence of SEQ ID NO: 30 (or an inhibitory oligonucleotide consisting of or consisting essentially of the nucleotide sequence of SEQ ID NO: 21 and an inhibitory oligonucleotide consisting of or consisting essentially of the nucleotide sequence of SEQ ID NO: 30).

In some embodiments, a nucleic acid vector (e.g., a recombinant viral genome) comprises an inhibitory oligonucleotide comprising a nucleotide sequence having at least 95% identity to the nucleotide sequence of any one of SEQ ID NOS: 47-82. In some embodiments, a nucleic acid vector (e.g., a recombinant viral genome) comprises an inhibitory oligonucleotide comprising the nucleotide sequence of any one of SEQ ID NOS: 47-82.

In some embodiments, a nucleic acid vector (e.g., a recombinant viral genome) comprises a nucleotide substitution at one or more of positions 1, 2, 3, 4, 5, 6, 7, 8, 9, 10, 11, 12, 13, 14, 15, 16, 17, 18,19, 20, 21, 22, 23, and 24, relative to the nucleotide sequence of SEQ ID NO: 2 (TTAGGGTTAGGGTTAGGGTTAGGG).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 47. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 47. In some embodiments, a nucleic acid vector (e.g., a recombinant viral genome) comprises an inhibitory oligonucleotide comprising the nucleotide sequence of SEQ ID NO: 47 (or an inhibitory oligonucleotide consisting of or consisting essentially of the nucleotide sequence of SEQ ID NO: 47). In some embodiments, a nucleic acid vector (e.g., a recombinant viral genome) comprises an inhibitory oligonucleotide comprising the nucleotide sequence of SEQ ID NO: 47 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 48. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 48. In some embodiments, a nucleic acid vector (e.g., a recombinant viral genome) comprises an inhibitory oligonucleotide comprising the nucleotide sequence of SEQ ID NO: 48 (or an inhibitory oligonucleotide consisting of or consisting essentially of the nucleotide sequence of SEQ ID NO: 48). In some embodiments, a nucleic acid vector (e.g., a recombinant viral genome) comprises an inhibitory oligonucleotide comprising the nucleotide sequence of SEQ ID NO: 48 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 49. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 49. In some embodiments, a nucleic acid vector (e.g., a recombinant viral genome) comprises an inhibitory oligonucleotide comprising the nucleotide sequence of SEQ ID NO: 49 (or an inhibitory oligonucleotide consisting of or consisting essentially of the nucleotide sequence of SEQ ID NO: 49). In some embodiments, a nucleic acid vector (e.g., a recombinant viral genome) comprises an inhibitory oligonucleotide comprising the nucleotide sequence of SEQ ID NO: 49 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 50. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 50. In some embodiments, a nucleic acid vector (e.g., a recombinant viral genome) comprises an inhibitory oligonucleotide comprising the nucleotide sequence of SEQ ID NO: 50 (or an inhibitory oligonucleotide consisting of or consisting essentially of the nucleotide sequence of SEQ ID NO: 50). In some embodiments, a nucleic acid vector (e.g., a recombinant viral genome) comprises an inhibitory oligonucleotide comprising the nucleotide sequence of SEQ ID NO: 50 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 51. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 51. In some embodiments, a nucleic acid vector (e.g., a recombinant viral genome) comprises an inhibitory oligonucleotide comprising the nucleotide sequence of SEQ ID NO: 51 (or an inhibitory oligonucleotide consisting of or consisting essentially of the nucleotide sequence of SEQ ID NO: 51). In some embodiments, a nucleic acid vector (e.g., a recombinant viral genome) comprises an inhibitory oligonucleotide comprising the nucleotide sequence of SEQ ID NO: 51 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 52. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 52. In some embodiments, a nucleic acid vector (e.g., a recombinant viral genome) comprises an inhibitory oligonucleotide comprising the nucleotide sequence of SEQ ID NO: 52 (or an inhibitory oligonucleotide consisting of or consisting essentially of the nucleotide sequence of SEQ ID NO: 52). In some embodiments, a nucleic acid vector (e.g., a recombinant viral genome) comprises an inhibitory oligonucleotide comprising the nucleotide sequence of SEQ ID NO: 52 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 53. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 53. In some embodiments, a nucleic acid vector (e.g., a recombinant viral genome) comprises an inhibitory oligonucleotide comprising the nucleotide sequence of SEQ ID NO: 53 (or an inhibitory oligonucleotide consisting of or consisting essentially of the nucleotide sequence of SEQ ID NO: 53). In some embodiments, a nucleic acid vector (e.g., a recombinant viral genome) comprises an inhibitory oligonucleotide comprising the nucleotide sequence of SEQ ID NO: 53 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 54. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 54. In some embodiments, a nucleic acid vector (e.g., a recombinant viral genome) comprises an inhibitory oligonucleotide comprising the nucleotide sequence of SEQ ID NO: 54 (or an inhibitory oligonucleotide consisting of or consisting essentially of the nucleotide sequence of SEQ ID NO: 54). In some embodiments, a nucleic acid vector (e.g., a recombinant viral genome) comprises an inhibitory oligonucleotide comprising the nucleotide sequence of SEQ ID NO: 54 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 55. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 55. In some embodiments, a nucleic acid vector (e.g., a recombinant viral genome) comprises an inhibitory oligonucleotide comprising the nucleotide sequence of SEQ ID NO: 55 (or an inhibitory oligonucleotide consisting of or consisting essentially of the nucleotide sequence of SEQ ID NO: 55). In some embodiments, a nucleic acid vector (e.g., a recombinant viral genome) comprises an inhibitory oligonucleotide comprising the nucleotide sequence of SEQ ID NO: 55 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 56. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 56. In some embodiments, a nucleic acid vector (e.g., a recombinant viral genome) comprises an inhibitory oligonucleotide comprising the nucleotide sequence of SEQ ID NO: 56 (or an inhibitory oligonucleotide consisting of or consisting essentially of the nucleotide sequence of SEQ ID NO: 56). In some embodiments, a nucleic acid vector (e.g., a recombinant viral genome) comprises an inhibitory oligonucleotide comprising the nucleotide sequence of SEQ ID NO: 56 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 57. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 57. In some embodiments, a nucleic acid vector (e.g., a recombinant viral genome) comprises an inhibitory oligonucleotide comprising the nucleotide sequence of SEQ ID NO: 57 (or an inhibitory oligonucleotide consisting of or consisting essentially of the nucleotide sequence of SEQ ID NO: 57). In some embodiments, a nucleic acid vector (e.g., a recombinant viral genome) comprises an inhibitory oligonucleotide comprising the nucleotide sequence of SEQ ID NO: 57 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 58. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 58. In some embodiments, a nucleic acid vector (e.g., a recombinant viral genome) comprises an inhibitory oligonucleotide comprising the nucleotide sequence of SEQ ID NO: 58 (or an inhibitory oligonucleotide consisting of or consisting essentially of the nucleotide sequence of SEQ ID NO: 58). In some embodiments, a nucleic acid vector (e.g., a recombinant viral genome) comprises an inhibitory oligonucleotide comprising the nucleotide sequence of SEQ ID NO: 58 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 59. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 59. In some embodiments, a nucleic acid vector (e.g., a recombinant viral genome) comprises an inhibitory oligonucleotide comprising the nucleotide sequence of SEQ ID NO: 59 (or an inhibitory oligonucleotide consisting of or consisting essentially of the nucleotide sequence of SEQ ID NO: 59). In some embodiments, a nucleic acid vector (e.g., a recombinant viral genome) comprises an inhibitory oligonucleotide comprising the nucleotide sequence of SEQ ID NO: 59 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 60. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 60. In some embodiments, a nucleic acid vector (e.g., a recombinant viral genome) comprises an inhibitory oligonucleotide comprising the nucleotide sequence of SEQ ID NO: 60 (or an inhibitory oligonucleotide consisting of or consisting essentially of the nucleotide sequence of SEQ ID NO: 60). In some embodiments, a nucleic acid vector (e.g., a recombinant viral genome) comprises an inhibitory oligonucleotide comprising the nucleotide sequence of SEQ ID NO: 60 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 61. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 61. In some embodiments, a nucleic acid vector (e.g., a recombinant viral genome) comprises an inhibitory oligonucleotide comprising the nucleotide sequence of SEQ ID NO: 61 (or an inhibitory oligonucleotide consisting of or consisting essentially of the nucleotide sequence of SEQ ID NO: 61). In some embodiments, a nucleic acid vector (e.g., a recombinant viral genome) comprises an inhibitory oligonucleotide comprising the nucleotide sequence of SEQ ID NO: 61 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 62. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 62. In some embodiments, a nucleic acid vector (e.g., a recombinant viral genome) comprises an inhibitory oligonucleotide comprising the nucleotide sequence of SEQ ID NO: 62 (or an inhibitory oligonucleotide consisting of or consisting essentially of the nucleotide sequence of SEQ ID NO: 62). In some embodiments, a nucleic acid vector (e.g., a recombinant viral genome) comprises an inhibitory oligonucleotide comprising the nucleotide sequence of SEQ ID NO: 62 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 63. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 63. In some embodiments, a nucleic acid vector (e.g., a recombinant viral genome) comprises an inhibitory oligonucleotide comprising the nucleotide sequence of SEQ ID NO: 63 (or an inhibitory oligonucleotide consisting of or consisting essentially of the nucleotide sequence of SEQ ID NO: 63). In some embodiments, a nucleic acid vector (e.g., a recombinant viral genome) comprises an inhibitory oligonucleotide comprising the nucleotide sequence of SEQ ID NO: 63 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 64. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 64. In some embodiments, a nucleic acid vector (e.g., a recombinant viral genome) comprises an inhibitory oligonucleotide comprising the nucleotide sequence of SEQ ID NO: 64 (or an inhibitory oligonucleotide consisting of or consisting essentially of the nucleotide sequence of SEQ ID NO: 64). In some embodiments, a nucleic acid vector (e.g., a recombinant viral genome) comprises an inhibitory oligonucleotide comprising the nucleotide sequence of SEQ ID NO: 64 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 65. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 65. In some embodiments, a nucleic acid vector (e.g., a recombinant viral genome) comprises an inhibitory oligonucleotide comprising the nucleotide sequence of SEQ ID NO: 65 (or an inhibitory oligonucleotide consisting of or consisting essentially of the nucleotide sequence of SEQ ID NO: 65). In some embodiments, a nucleic acid vector (e.g., a recombinant viral genome) comprises an inhibitory oligonucleotide comprising the nucleotide sequence of SEQ ID NO: 65 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 66. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 66. In some embodiments, a nucleic acid vector (e.g., a recombinant viral genome) comprises an inhibitory oligonucleotide comprising the nucleotide sequence of SEQ ID NO: 66 (or an inhibitory oligonucleotide consisting of or consisting essentially of the nucleotide sequence of SEQ ID NO: 66). In some embodiments, a nucleic acid vector (e.g., a recombinant viral genome) comprises an inhibitory oligonucleotide comprising the nucleotide sequence of SEQ ID NO: 66 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 67. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 67. In some embodiments, a nucleic acid vector (e.g., a recombinant viral genome) comprises an inhibitory oligonucleotide comprising the nucleotide sequence of SEQ ID NO: 67 (or an inhibitory oligonucleotide consisting of or consisting essentially of the nucleotide sequence of SEQ ID NO: 67). In some embodiments, a nucleic acid vector (e.g., a recombinant viral genome) comprises an inhibitory oligonucleotide comprising the nucleotide sequence of SEQ ID NO: 67 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 68. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 68. In some embodiments, a nucleic acid vector (e.g., a recombinant viral genome) comprises an inhibitory oligonucleotide comprising the nucleotide sequence of SEQ ID NO: 68 (or an inhibitory oligonucleotide consisting of or consisting essentially of the nucleotide sequence of SEQ ID NO: 68). In some embodiments, a nucleic acid vector (e.g., a recombinant viral genome) comprises an inhibitory oligonucleotide comprising the nucleotide sequence of SEQ ID NO: 68 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 69. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 69. In some embodiments, a nucleic acid vector (e.g., a recombinant viral genome) comprises an inhibitory oligonucleotide comprising the nucleotide sequence of SEQ ID NO: 69 (or an inhibitory oligonucleotide consisting of or consisting essentially of the nucleotide sequence of SEQ ID NO: 69). In some embodiments, a nucleic acid vector (e.g., a recombinant viral genome) comprises an inhibitory oligonucleotide comprising the nucleotide sequence of SEQ ID NO: 69 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 70. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 70. In some embodiments, a nucleic acid vector (e.g., a recombinant viral genome) comprises an inhibitory oligonucleotide comprising the nucleotide sequence of SEQ ID NO: 70 (or an inhibitory oligonucleotide consisting of or consisting essentially of the nucleotide sequence of SEQ ID NO: 70). In some embodiments, a nucleic acid vector (e.g., a recombinant viral genome) comprises an inhibitory oligonucleotide comprising the nucleotide sequence of SEQ ID NO: 70 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 71. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 71. In some embodiments, a nucleic acid vector (e.g., a recombinant viral genome) comprises an inhibitory oligonucleotide comprising the nucleotide sequence of SEQ ID NO: 71 (or an inhibitory oligonucleotide consisting of or consisting essentially of the nucleotide sequence of SEQ ID NO: 71). In some embodiments, a nucleic acid vector (e.g., a recombinant viral genome) comprises an inhibitory oligonucleotide comprising the nucleotide sequence of SEQ ID NO: 71 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 72. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 72. In some embodiments, a nucleic acid vector (e.g., a recombinant viral genome) comprises an inhibitory oligonucleotide comprising the nucleotide sequence of SEQ ID NO: 72 (or an inhibitory oligonucleotide consisting of or consisting essentially of the nucleotide sequence of SEQ ID NO: 72). In some embodiments, a nucleic acid vector (e.g., a recombinant viral genome) comprises an inhibitory oligonucleotide comprising the nucleotide sequence of SEQ ID NO: 72 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 73. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 73. In some embodiments, a nucleic acid vector (e.g., a recombinant viral genome) comprises an inhibitory oligonucleotide comprising the nucleotide sequence of SEQ ID NO: 73 (or an inhibitory oligonucleotide consisting of or consisting essentially of the nucleotide sequence of SEQ ID NO: 73). In some embodiments, a nucleic acid vector (e.g., a recombinant viral genome) comprises an inhibitory oligonucleotide comprising the nucleotide sequence of SEQ ID NO: 73 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 74. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 74. In some embodiments, a nucleic acid vector (e.g., a recombinant viral genome) comprises an inhibitory oligonucleotide comprising the nucleotide sequence of SEQ ID NO: 74 (or an inhibitory oligonucleotide consisting of or consisting essentially of the nucleotide sequence of SEQ ID NO: 74). In some embodiments, a nucleic acid vector (e.g., a recombinant viral genome) comprises an inhibitory oligonucleotide comprising the nucleotide sequence of SEQ ID NO: 74 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 75. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 75. In some embodiments, a nucleic acid vector (e.g., a recombinant viral genome) comprises an inhibitory oligonucleotide comprising the nucleotide sequence of SEQ ID NO: 75 (or an inhibitory oligonucleotide consisting of or consisting essentially of the nucleotide sequence of SEQ ID NO: 75). In some embodiments, a nucleic acid vector (e.g., a recombinant viral genome) comprises an inhibitory oligonucleotide comprising the nucleotide sequence of SEQ ID NO: 75 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 76. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 76. In some embodiments, a nucleic acid vector (e.g., a recombinant viral genome) comprises an inhibitory oligonucleotide comprising the nucleotide sequence of SEQ ID NO: 76 (or an inhibitory oligonucleotide consisting of or consisting essentially of the nucleotide sequence of SEQ ID NO: 76). In some embodiments, a nucleic acid vector (e.g., a recombinant viral genome) comprises an inhibitory oligonucleotide comprising the nucleotide sequence of SEQ ID NO: 76 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 77. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 77. In some embodiments, a nucleic acid vector (e.g., a recombinant viral genome) comprises an inhibitory oligonucleotide comprising the nucleotide sequence of SEQ ID NO: 77 (or an inhibitory oligonucleotide consisting of or consisting essentially of the nucleotide sequence of SEQ ID NO: 77). In some embodiments, a nucleic acid vector (e.g., a recombinant viral genome) comprises an inhibitory oligonucleotide comprising the nucleotide sequence of SEQ ID NO: 77 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 78. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 78. In some embodiments, a nucleic acid vector (e.g., a recombinant viral genome) comprises an inhibitory oligonucleotide comprising the nucleotide sequence of SEQ ID NO: 78 (or an inhibitory oligonucleotide consisting of or consisting essentially of the nucleotide sequence of SEQ ID NO: 78). In some embodiments, a nucleic acid vector (e.g., a recombinant viral genome) comprises an inhibitory oligonucleotide comprising the nucleotide sequence of SEQ ID NO: 78 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 79. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 79. In some embodiments, a nucleic acid vector (e.g., a recombinant viral genome) comprises an inhibitory oligonucleotide comprising the nucleotide sequence of SEQ ID NO: 79 (or an inhibitory oligonucleotide consisting of or consisting essentially of the nucleotide sequence of SEQ ID NO: 79). In some embodiments, a nucleic acid vector (e.g., a recombinant viral genome) comprises an inhibitory oligonucleotide comprising the nucleotide sequence of SEQ ID NO: 79 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 80. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 80. In some embodiments, a nucleic acid vector (e.g., a recombinant viral genome) comprises an inhibitory oligonucleotide comprising the nucleotide sequence of SEQ ID NO: 80 (or an inhibitory oligonucleotide consisting of or consisting essentially of the nucleotide sequence of SEQ ID NO: 80). In some embodiments, a nucleic acid vector (e.g., a recombinant viral genome) comprises an inhibitory oligonucleotide comprising the nucleotide sequence of SEQ ID NO: 80 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 81. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 81. In some embodiments, a nucleic acid vector (e.g., a recombinant viral genome) comprises an inhibitory oligonucleotide comprising the nucleotide sequence of SEQ ID NO: 81 (or an inhibitory oligonucleotide consisting of or consisting essentially of the nucleotide sequence of SEQ ID NO: 81). In some embodiments, a nucleic acid vector (e.g., a recombinant viral genome) comprises an inhibitory oligonucleotide comprising the nucleotide sequence of SEQ ID NO: 81 and a therapeutic nucleotide sequence (e.g., a therapeutic DNA, a therapeutic RNA, or a nucleotide sequence encoding a therapeutic protein).

In some embodiments, a nucleic acid vector (e.g., a recombinant viral genome) comprises an inhibitory oligonucleotide comprising a nucleotide sequence having at least 95% identity to the nucleotide sequence of SEQ ID NO: 82. In some embodiments, a nucleic acid vector (e.g., a recombinant viral genome) comprises an inhibitory oligonucleotide comprising a nucleotide sequence having no more than 1 nucleotide substitution, relative to the nucleotide sequence of SEQ ID NO: 82. In some embodiments, a nucleic acid vector (e.g., a recombinant viral genome) comprises an inhibitory oligonucleotide comprising the nucleotide sequence of SEQ ID NO: 82 (or an inhibitory oligonucleotide consisting of or consisting essentially of the nucleotide sequence of SEQ ID NO: 82). In some embodiments, a nucleic acid vector (e.g., a recombinant viral genome) comprises an inhibitory oligonucleotide comprising the nucleotide sequence of SEQ ID NO: 82 and a therapeutic nucleotide sequence (e.g., a therapeutic DNA, a therapeutic RNA, or a nucleotide sequence encoding a therapeutic protein).

It should be understood that a nucleic acid vector (e.g., a recombinant viral genome) may include any two or more (2, 3, 4, 5, 6, 7, 8, 9, 10, or more) inhibitory molecules, for example, selected from any one of SEQ ID NOS: 4-40 and 47-82.

### Recombinant Viral Genomes

The present disclosure provides recombinant viral genomes in which an (at least one) inhibitory oligonucleotide is included in the viral genome. In some embodiments, two or more inhibitory oligonucleotides are included in the viral genome. Multiple inhibitory oligonucleotides may be located in different locations throughout the viral genome (relative to each other). The viral genomes typically include a therapeutic nucleotide sequence and an inhibitory oligonucleotide. The inhibitory oligonucleotide may be located, for example, in the 3′ untranslated region (UTR) of the viral genome. In some embodiments, the inhibitory oligonucleotide is downstream (3′) relative to the therapeutic nucleotide sequence. In some embodiments, the inhibitory oligonucleotide is located downstream from (3′) a polyA tail linked to the therapeutic nucleotide sequence. In some embodiments, the inhibitory oligonucleotide is located in the 5′ UTR of the viral genome. In some embodiments, the inhibitory oligonucleotide is located upstream (5′) of a promoter operably linked to the therapeutic nucleotide sequence. In some instances, the inhibitory oligonucleotide is located upstream (5′) relative to the therapeutic nucleotide sequence. As a non-limiting example, a recombinant viral genome may comprise an inhibitory oligonucleotide located downstream (3′) (e.g., downstream from (3′) a polyA tail linked to the therapeutic nucleotide sequence, including in the 3′ UTR) and an inhibitory oligonucleotide located upstream (5′) (e.g., in the 5′ UTR) of the therapeutic nucleotide sequence. Surprisingly, the location of the inhibitory oligonucleotide in the viral genome, relative to the promoter, does not impact the inhibitory function of the oligonucleotide. The recombinant viral genome may comprise inflammatory nucleic acids. The inflammatory nucleic acids may be located anywhere in the viral genome (e.g., the viral ITR, the promoter, the intron, the transgene, the 5′ UTR, the 3′ UTR, etc.). For example, the therapeutic nucleotide sequence may comprise inflammatory nucleic acids.

The inhibitory oligonucleotides of the present disclosure may be oriented in the sense direction and/or antisense direction in the viral genome. In some embodiments, a viral genome includes 1, 2, 3, 4, or 5 copies of an inhibitory oligonucleotide in the sense direction. In some embodiments, the same viral genome includes 1, 2, 3, 4, or 5 copies of the same inhibitory oligonucleotide or a different oligonucleotide in the antisense direction.

The recombinant viral genomes, as provided herein, may be used, in some embodiments, to deliver (to a subject) a therapeutic nucleotide sequence of interest (e.g., a therapeutic DNA, a therapeutic RNA, and/or a therapeutic protein encoded by the nucleotide sequence). In some embodiments, the recombinant viral genomes of the present disclosure are gene delivery vectors. Thus, in some embodiments, the therapeutic nucleotide sequence is a gene encoding a therapeutic protein, as discussed elsewhere herein.

A recombinant viral genome, generally, is a viral genome that is not naturally occurring. The viral genomes may be from adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B19. Other viral genomes are encompassed by the present disclosure.

In some embodiments, a viral genome is an AAV genome. AAV is a small, non-enveloped virus that packages a single-stranded linear DNA genome that is approximately 5 kb long, and has been adapted for use as a gene transfer vehicle (Samulski, R J et al., Annu Rev Virol. 2014;1(1):427-51). The coding regions of AAV are flanked by inverted terminal repeats (ITRs), which act as the origins for DNA replication and serve as the primary packaging signal (McLaughlin, S K et al. J Virol. 1988;62(6):1963-73; Hauswirth, W W et al. 1977;78(2):488-99). Both positive and negative strands are packaged into virions equally well and capable of infection (Zhong, Let al. Mol Ther. 2008;16(2):290-5; Zhou, X et al. Mol Ther. 2008;16(3):494-9; Samulski, R J et al. J Virol. 1987;61(10):3096-101). In addition, a small deletion in one of the two ITRs allows packaging of self-complementary vectors, in which the genome self-anneals after viral uncoating. This results in more efficient transduction of cells but reduces the coding capacity by half (McCarty, D M et al. Mol Ther. 2008;16(10):1648-56; McCarty, D M et al. Gene Ther. 2001;8(16):1248-54).

In some embodiments, the recombinant viral genomes of the present disclosure include a single-stranded nucleotide sequence. In some embodiments, the viral genome is self-complementary. A self-complementary viral genome is a viral genome that forms an intramolecular double-stranded nucleotide sequence.

In some embodiments, the viral genome is a single-stranded nucleotide sequence (e.g., ssAAV). In some embodiments, the single-stranded viral genome does not form an intramolecular double-stranded nucleotide sequence. In some embodiments, a recombinant viral genome is a single-stranded viral genome comprising an inhibitory oligonucleotide downstream (3′) of the therapeutic nucleotide sequence (e.g., downstream from (3′) a polyA tail linked to the therapeutic nucleotide sequence, including in the 3′ UTR of the viral genome). In some embodiments, a recombinant viral genome is a single-stranded viral genome comprising an inhibitory oligonucleotide upstream (5′) of the therapeutic nucleotide sequence (e.g., in the 5′ UTR of the viral genome). In some embodiments, a recombinant viral genome is a single-stranded viral genome comprising an inhibitory oligonucleotide upstream (5′) of the therapeutic nucleotide sequence (e.g., in the 5′ UTR of the viral genome) and an inhibitory oligonucleotide downstream (3′) of the therapeutic nucleotide sequence (e.g., in the 3′ UTR of the viral genome).

As a non-limiting example, a recombinant viral genome may comprise at least two different inhibitory oligonucleotides located upstream (5′) of a therapeutic nucleotide sequence and at least two different inhibitory oligonucleotides sequences located downstream (3′) of the therapeutic nucleotide sequence. In some instances, all inhibitory oligonucleotides sequences are different in a recombinant viral genome. For example, a recombinant viral genome may comprise inhibitory oligonucleotides that each comprises different multiple tandem repeats of a sequence that is at least 90% identical to a sequence of any one of SEQ ID NOS: 4-40. In some instances, an inhibitory oligonucleotide comprises multiple tandem repeats of two different sequences selected from to a sequence of any one of SEQ ID NOS: 4-40. As another example, a recombinant viral genome may comprise inhibitory oligonucleotides that each comprises different multiple tandem repeats of a sequence that is at least 90% identical to a sequence of any one of SEQ ID NOS: 47-82. In some instances, an inhibitory oligonucleotide comprises multiple tandem repeats of two different sequences selected from to a sequence of any one of SEQ ID NOS: 47-82.

### Therapeutic Nucleotide Sequences

The nucleic acid vectors (e.g., a recombinant viral genome) of the present disclosure may comprise a therapeutic nucleotide sequence. A therapeutic nucleotide sequence is a nucleotide sequence (e.g., RNA or DNA) that confers a therapeutic benefit or encodes a molecule (e.g., protein) that confers a therapeutic benefit to a subject when administered in vivo. In some embodiments, the therapeutic nucleotide sequence is a therapeutic RNA sequence (e.g., an RNAi molecule). In some embodiments, the therapeutic nucleotide sequence is a therapeutic DNA sequence (e.g. a DNA aptamer that binds a target). In some embodiments, the therapeutic nucleotide sequence encodes a therapeutic protein or peptide. For example, the therapeutic nucleotide sequence may encode a wild-type (unmodified) protein to compensate for a modified (e.g., mutated or truncated) version of the protein present in a subject or to compensate for a protein the subject lacks. Non-limiting examples of proteins encoded by a therapeutic nucleotide sequence include antibodies, enzymes, hormones, growth factors, cytokines and fusion proteins.

In some embodiments, the therapeutic nucleotide sequence is configured to replace a disease allele. For example, the therapeutic nucleotide sequence could be designed to facilitate nonhomologous end joining or homologous recombination. In some embodiments, the therapeutic gene sequence is a programmable nuclease. Non limiting examples of programmable nucleases include Cas9, Cpf1, C2c2, zinc finger, zinc finger nucleases, TALEs, TALENs, meganucleases, and fusions thereof to effector domains. Effector domains include transcriptional activators, transcription repressors, transposes, recombinases and deaminases. In some embodiments, the therapeutic nucleotide sequence encodes a guide RNA (e.g., for gene editing) or a DNA template (e.g., for homologous recombination).

In some examples, the therapeutic nucleotide sequence itself is a therapeutic molecule. In some embodiments, the nucleotide sequence is a DNA aptamer that binds a molecular target (e.g., protein target). A process termed SELEX (systematic evolution of ligands by exponential enrichment) is frequently used to select oligonucleotides from a DNA library that bind strongly to a target (Zhou J et al. Ther Nucleic Acids. 2014;3:e169). Examples of DNA aptamers include AS1411, which binds to the cellular protein nucleolin and has been tested as an anticancer agent (Bates P J et al. Exp Mol Pathol. 2009;86(3):151-64; Soundararajan S et al. Cancer Res. 2008;68(7):2358-65), and ARC1779, a PEGylated DNA aptamer which binds to von Willebrand factor to inhibit its interaction with platelets, thereby inducing an antithrombotic effect (Markus H S et al. Stroke. 2011;42(8):2149-53).

In some embodiments, the therapeutic nucleotide sequence encodes a sequence that is capable of reducing expression of a disease gene. In some embodiments, the therapeutic nucleotide sequence is complementary to a mRNA encoding a disease gene. As a non-limiting example, the therapeutic nucleotide sequence may be a guide RNA (e.g., for use in CRISPR systems), a siRNA, a microRNA (miRNA), or a short hairpin RNA (shRNA). In some embodiments, a therapeutic nucleotide sequence targets a mutant allele.

In some embodiments, the nucleic acid vector (e.g., a recombinant viral genome) comprises a promoter operably linked to the therapeutic nucleotide sequence. A promoter is a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. A promoter drives expression or drives transcription of the nucleic acid sequence that it regulates. A promoter is considered to be ‘operably linked’ to a nucleotide sequence when it is in a correct functional location and orientation in relation to the nucleotide sequence to control (‘drive’) transcriptional initiation and/or expression of that sequence. Promoters may be constitutive or inducible. An inducible promoter is a promoter that is regulated (e.g., activated or inactivated) by the presence or absence of a particular factor.

In some instances, a suitable host cell line (e.g., HEK293T, HeLa cells and Sf9 insect cells) may be used for producing viral particles encoding the nucleic acid vectors (e.g., a recombinant viral genome) disclosed herein following routine practice. One or more expression vectors (e.g. viral vectors) encoding viral components, at least one therapeutic nucleotide sequence and at least one inhibitory oligonucleotide described herein may be introduced into the suitable host cells, which can then be cultured under suitable conditions allowing for production of the viral particles. When needed, a helper virus can be used to facilitate replication and/or assembly of the viral particles. Alternatively, a host cell line producing one or more of essential viral components for viral genome replication and/or viral particle assembly may be used. The supernatant of the cell culture may be collected and the viral particles contained therein can be collected via routine methodology. As an example, a method for AAV production is provided in the Materials and Methods section below.

The nucleic acid vectors (e.g., a recombinant viral genome) as provided herein may be administered by intravenous, intramuscular, subretinal, intravitreal, intrathecal, intraparenchymal, and intracranial injections. In some embodiments, the nucleic acid vectors (e.g., a recombinant viral genome) are delivered by intramuscular injection. In some embodiments, the nucleic acid vectors (e.g., a recombinant viral genome) are delivered by intravenous injection.

In some embodiments, the nucleic acid vectors (e.g., a recombinant viral genome) are used to transduce cells in the liver, skeletal muscle, cardiac muscle, eye (e.g., retina), central nervous system or any combination thereof.

### Pharmaceutical Compositions

In some aspects, the present disclosure provides compositions comprising any of the nucleic acid vectors (e.g., a recombinant viral genomes) as disclosed herein. In some embodiments, the compositions further comprise a pharmaceutically-acceptable excipient. Non-limiting examples of pharmaceutically-acceptable excipients include water, saline, dextrose, glycerol, ethanol and combinations thereof. The excipient may be selected on the basis of the mode and route of administration, and standard pharmaceutical practice.

Nucleic acid vectors (e.g., a recombinant viral genomes), in some embodiments, may be formulated in a delivery vehicle. Non-limiting examples of delivery vehicles include nanoparticles, such as nanocapsules and nanospheres. See, e.g., Sing, R et al. Exp Mol Pathol. 2009;86(3):215-223. A nanocapsule is often comprised of a polymeric shell encapsulating a drug (e.g., recombinant viral genome of the present disclosure). Nanospheres are often comprised of a solid polymeric matrix throughout which the drug (e.g. recombinant viral genome) is dispersed. In some embodiments, the nanoparticle is a lipid particle, such as a liposome. See, e.g., Puri, A et al. Crit Rev Ther Drug Carrier Syst. 2009;26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283,1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the nucleic acid vectors (e.g., a recombinant viral genomes) disclosed herein may be found, for example, in Remington's Pharmaceutical Sciences, 18th edition, Mack Publishing Co., Easton, Pa. (1990) (incorporated herein by reference in its entirety).

### Methods of Delivery

Any of the nucleic acid vectors (e.g., a recombinant viral genomes) or compositions disclosed herein may be administered to a subject (e.g., mammalian subject, such as a human, mouse, rabbit, goat. sheep or pig) to inhibit the inflammatory response (e.g., inhibit induction of the inflammatory response). In some embodiments, the subject is in need of gene therapy. For example, the subject may have a genetic disorder (e.g., characterized by chromosomal abnormality and/or gene defects including mutation, truncation, insertion and deletion). The subject may have, may be suspected of having, or may at risk for a disease. In some embodiments, the disease is an ocular disease. As used herein, an “ocular disease” or “eye disease” is a disease or condition of the eye (e.g., retinal disease). Non-limiting examples of conditions that affect the eye include Stargardt disease, Ectropion, Lagophthalmos, Blepharochalasis, Ptosis, Stye, Xanthelasma, Dermatitis, Demodex, leishmaniasis, loiasis, onchocerciasis, phthiriasis, (herpes simplex), leprosy, molluscum contagiosum, tuberculosis, yaws, zoster, impetigo, Dacryoadenitis, Epiphora, exophthalmos, Conjunctivitis, Scleritis, Keratitis, Corneal ulcer/Corneal abrasion, Snow blindness/Arc eye, Thygeson's superficial punctate keratopathy, Corneal neovascularization, Fuchs' dystrophy, Keratoconus, Keratoconjunctivitis sicca, Iritis, iris, Uveitis, Sympathetic ophthalmia, Cataract, Chorioretinal inflammation, Focal chorioretinal inflammation, chorioretinitis, choroiditis, retinitis, retinochoroiditis, Disseminated chorioretinal inflammation, exudative retinopathy, Posterior cyclitis, Pars planitis, chorioretinal inflammations, Harada's disease, Chorioretinal inflammation, choroid, Chorioretinal scars, Macula scars, posterior pole (postinflammatory) (post-traumatic), Solar retinopathy, Choroidal degeneration, Atrophy, Sclerosis, angioid streaks, choroidal dystrophy, Choroideremia, choroidal, areolar, (peripapillary), Gyrate atrophy, choroid, ornithinaemia, Choroidal haemorrhage, Choroidal haemorrhage, NOS (Not Otherwise Specified), Choroidal detachment, Chorioretinal, Chorioretinal inflammation, infectious and parasitic diseases, Chorioretinitis, syphilitic, toxoplasma, tuberculosis, chorioretinal, Retinal detachment, distorted vision, Retinoschisis, Hypertensive retinopathy, Diabetic retinopathy, Retinopathy, Retinopathy of prematurity, Age-related macular degeneration, macula, Macular degeneration, Bull's Eye Maculopathy, Epiretinal membrane, Peripheral retinal degeneration, Hereditary retinal dystrophy, Retinitis pigmentosa, Retinal haemorrhage, retinal layers, Central serous retinopathy, Retinal detachment, retinal disorders, Macular edema, macula, Retinal disorder, Diabetic retinopathy, Glaucoma, optic neuropathy, ocular hypertension, open-angle glaucoma, angle-closure glaucoma, Normal Tension glaucoma, open-angle glaucoma, angle-closure glaucoma, Floaters, Leber's hereditary optic neuropathy, Optic disc drusen, Strabismus, Ophthalmoparesis, eye muscles, Progressive external ophthaloplegia, Esotropia, Exotropia, Disorders of refraction, accommodation, Hypermetropia, Myopia, Astigmatism, Anisometropia, Presbyopia, ophthalmoplegia, Amblyopia, Leber's congenital amaurosis, Scotoma, Anopsia, Color blindness, Achromatopsia/Maskun, Nyctalopia, Blindness, River blindness, Micropthalmia/coloboma, Red eye, Argyll Robertson pupil, pupils, Keratomycosis, Xerophthalmia, and Aniridia.

In some embodiments, the disease affects muscle. Non-limiting examples of muscle diseases include Pompe disease, Barth syndrome, Duchenne muscular dystrophy, Becker muscular dystrophy, myotonic dystrophy, facioscapulohumeral muscular dystrophy, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, and rhabdomyolysis.

In some embodiments, the disease affects the central nervous system (CNS). Non-limiting examples of CNS diseases include Friedreich ataxia, Batten disease, Parkinson's disease, giant axon neuropathy (GAN), Canavan disease, mucopolysaccharidosis (types Ito VII), metachromatic leukodystrophy (MLD), and spinal muscular atrophy (SMA).

In some embodiments, the disease is a metabolic disease, e.g., phenylketonuria (PKU). In some embodiments, the disease is a hemophilia, e.g., hemophilia A, hemophiliaB, or hemophilia C.

In some embodiments, the disease is a hemoglobinopathy, e.g., α-thalassemia, β-thalassemia, or sickle cell disease.

Suitable routes of administration include parenterally, by injection, for example, intravenously, subcutaneously, intramuscularly intrathecally, intraperitoneally, intraparenchymal, intracuteanously, intrasternally, intraarticularlly, intracranially, intralesionally, intrarectually, intravaginally, intranasally, intragastically, intratracheally, or intrapulmonarily. Alternatively, other modes of administration including suppositories, oral formulations, enteral, nasal, topical or transmucosal administration may be desirable. Oral formulations may include normally employed excipients such as, for example, pharmaceutical grades of saccharine, cellulose, magnesium carbonate and the like. These compositions may take the form of solutions, suspensions, tablets, pills, capsules, sustained release formulations or powders.

In some embodiments, a recombinant viral genome comprising an inhibitory oligonucleotide is administered intramuscularly. In some embodiments, the intramuscularly administered recombinant viral genome comprises an adeno-associated viral genome, which comprises a therapeutic nucleotide sequence and an inhibitory oligonucleotide. In some embodiments, the inhibitory oligonucleotide comprises a nucleotide sequence of any one of SEQ ID NOS: 4-40. In some embodiments, the inhibitory oligonucleotide comprises a nucleotide sequence of any one of SEQ ID NOS: 47-82. In some embodiments, the viral genome administered intramuscularly is expressed in muscle cells.

In some embodiments, the nucleic acid vectors (e.g., a recombinant viral genomes) of the present disclosure are administered intravenously to a subject. In some embodiments, the nucleic acid vectors (e.g., a recombinant viral genomes) are administered peritoneally to a subject. In some embodiments, the intravenously or peritoneally administered recombinant viral genome comprises an adeno-associated viral genome, which includes a therapeutic nucleotide sequence and an inhibitory nucleotide sequence. In some embodiments, the inhibitory nucleotide sequence comprises a nucleotide sequence of any one of SEQ ID NOS: 4-40. In some embodiments, the inhibitory oligonucleotide comprises a nucleotide sequence of any one of SEQ ID NOS: 47-82. In some embodiments, the recombinant viral genome administered intravenously or peritoneally is expressed in the liver cells of a subject.

An inflammatory response (e.g., local or systemic) may be assessed by measuring the level of cytokine activity and/or expression in a subject. In some embodiments, the level of expression and/or activity of IL-6, TNF, interferon (e.g., IFNα, IFNβ, and IFNγ), and/or CXCL10 is measured. Typically, the level of cytokine expression and/or activity correlates with the degree of the inflammatory response. Thus, a subject who has received a recombinant viral genome of the present disclosure (comprising a therapeutic nucleotide sequence and an inhibitory oligonucleotide) may have a reduction of or undetectable expression and/or activity levels of certain cytokines, indicative of a reduced or no inflammatory response, compared to a subject who has received a recombinant viral genome that does not include an inhibitory oligonucleotide. In some embodiments, the control inflammatory response for comparison is the inflammatory response elicited by a viral genome that does not comprise an inhibitory oligonucleotide as determined by the same or a substantially similar assay under the same or substantially similar conditions.

In some embodiments, a recombinant viral genome of the present disclosure elicits an inflammatory response in the subject that is at least 2-fold lower than a control. For example, a recombinant viral genome may elicit an inflammatory response in the subject that is at least 3-fold, 4-fold, 5-fold, 6-fold, 7-fold, 8-fold, 9-fold, 10-fold, 25-fold or 50-fold lower than a control. In some embodiments, a recombinant viral genome elicits an inflammatory response in the subject that is at least 10% lower than a control. For example, a recombinant viral genome may elicit an inflammatory response in the subject that is at least 15%, 20%, 25%, 30%, 35%, 40%, 45%, 50%, 55%, 60%, 65%, 70%, 75%, 80%, 85%, 90%, or 95% lower than a control. In some embodiments, a recombinant viral genome is expressed in cells of the subject at a level that is 15% to 100%, 20% to 100%, 25% to 100%, 50% to 100%, 60% to 100%, 70% to 100%, 80% to 100%, or 90% to 100% higher than a control. In some embodiments, a recombinant viral genome may inhibit induction of an inflammatory response compared to a control, such that the inflammatory response is undetectable. A control, in some embodiments, is an inflammatory response elicited in a subject by a viral genome that does not comprise an inhibitory oligonucleotide.

In some embodiments, a recombinant viral genome of the present disclosure reduces AAV-induced pathology in the eye of a subject compared to control. A control, in some embodiments, is the pathology, including tissue damage and alteration in morphology, elicited in an organ by a viral genome that does not comprise an inhibitory oligonucleotide. For example, a recombinant viral genome of the present disclosure may reduce loss of cone outer segments, reduce shortening of cone outer segments, or alter the morphology of cone outer segments less than a control. For example, an eye receiving a recombinant viral genome of the present disclosure may have better preservation of cone outer segments and appeared morphologically closer to an eye receiving no viral genome.

Morphology of the eye or tissues of the eye can be determined using, for example, cone arrestin staining and opsin staining. Retinal images from in vivo optical coherence tomography (OCT) b-scans may be used to determine damage to outer retinal lamination. As a non-limiting example, the lengths of various types of damage (e.g., retinal detachment, non-severe laminar disruption, or severe laminar damage) on the optical coherence tomography (OCT) b-scans may be measured. In some instances, a recombinant viral genome of the present disclosure elicits less or no severe laminar damage compared to a viral genome without an inhibitory oligonucleotide.

In some embodiments, a recombinant viral genome (and thus the therapeutic nucleotide sequence of the recombinant viral genome) of the present disclosure is expressed in cells of the subject at a level that is at least 2-fold greater than a control. For example, a recombinant viral genome may be expressed in cells of the subject at a level that is at least 3-fold, 4-fold, 5-fold, 6-fold, 7-fold, 8-fold, 9-fold, 10-fold, 25-fold or 50-fold higher than a control. In some embodiments, a recombinant viral genome is expressed in cells of the subject at a level that is 2-fold to 50-fold, 5-fold to 50-fold, 10-fold to 50-fold, or 25-fold to 50-fold higher than a control. In some embodiments, a recombinant viral genome of the present disclosure is expressed in cells of the subject at a level that is at least 10% higher than a control. For example, a recombinant viral genome may be expressed in cells of the subject at a level that is at least 15%, 20%, 25%, 30%, 35%, 40%, 45%, 50%, 55%, 60%, 70%, 80% or 90% higher than a control. In some embodiments, a recombinant viral genome is expressed in cells of the subject at a level that is 15% to 100%, 20% to 100%, 25% to 100%, 50% to 100%, 60% to 100%, 70% to 100%, 80% to 100%, or 90% to 100% higher than a control. A control, in some embodiments, is the expression level of a viral genome that does not comprise an inhibitory oligonucleotide.

In some embodiments, a therapeutically effective amount of a recombinant viral genome of the present disclosure is administered to a subject to treat a genetic disorder, such as a muscle disorder or a liver disorder. A therapeutically effective amount, in some embodiments, is an amount of a therapeutic nucleotide sequence (and/or a nucleic acid vector, such as a recombinant viral genome) required to confer therapeutic effect on the subject. In some embodiments, a therapeutically effective amount is an amount of inhibitory oligonucleotide required to inhibit induction of an inflammatory response following administration of a recombinant viral genome (comprising a therapeutic nucleotide sequence and an inhibitor oligonucleotide) of the present disclosure. Effective amounts vary, as recognized by those skilled in the art, depending on the route of administration, excipient usage, and co-usage with other active agents. Effective amounts depend on the subject to be treated, including, for example, the weight, sex and age of the subject as well as the strength of the subject's immune system and/or genetic predisposition. Suitable dosage ranges are readily determinable by one skilled in the art. The effective amount (and thus the dosage and/or dosing schedule) of the compositions disclosed herein may also depend on the type of the viral genome, the type of therapeutic nucleotide sequence, and/or the type of inhibitory oligonucleotide.

In some embodiments, the therapeutically effective amount of a recombinant viral genome of the present disclosure is at least 20% lower than the therapeutically effective amount of a viral genome not comprising an inhibitory oligonucleotide. For example, the therapeutically effective amount of a recombinant viral genome of the present disclosure may be at least at least 25%, 30%, 40%, 50% or 60% (but less than 100%) lower than the therapeutically effective amount of a viral genome not comprising an inhibitory oligonucleotide. In some embodiments, administration of a recombinant viral genome of the present disclosure at a reduced therapeutically effective amount results in expression of an encoded therapeutic molecule at a level that is equal to or greater than (e.g., at least 5%, 10%, 20%, 30%, 40%, or 50% greater than) expression of the same encoded therapeutic molecule from a viral genome not comprising an inhibitory oligonucleotide.

In some embodiments, a method comprises administering to a subject a recombinant viral genome comprising the inhibitory nucleotide sequence of SEQ ID NO: 21, the inhibitory nucleotide sequence of SEQ ID NO: 30, and a nucleotide sequence encoding a therapeutic molecule. In some embodiments, a method comprises administering to a subject a recombinant viral genome comprising an inhibitory nucleotide sequence that consists of or consists essentially of the inhibitory nucleotide sequence of SEQ ID NO: 21, an inhibitory nucleotide sequence that consists of or consists essentially of the inhibitory nucleotide sequence of SEQ ID NO: 30, and a nucleotide sequence encoding a therapeutic molecule.

In some embodiments, a method comprises administering to a subject a recombinant viral genome comprising the inhibitory nucleotide sequence of SEQ ID NO: 40 and a nucleotide sequence encoding a therapeutic molecule. In some embodiments, a method comprises administering to a subject a recombinant viral genome comprising an inhibitory nucleotide sequence that consists of or consists essentially of the inhibitory nucleotide sequence of SEQ ID NO: 40, and a nucleotide sequence encoding a therapeutic molecule. In some embodiments, NK-κB activation in the subject is reduced by at least 90% or at least 98%, relative to a control (e.g., NK-κB activation in the subject administered a recombinant viral genome without an inhibitory nucleotide sequence of the present disclosure).

In some embodiments, a method comprises administering to a subject a recombinant viral genome comprising (a) an inhibitor oligonucleotide consisting of the nucleotide sequence of SEQ ID NO: 43 and (b) a nucleotide sequence encoding a therapeutic molecule. In some embodiments, a method comprises administering to a subject a recombinant viral genome comprising (a) an inhibitor oligonucleotide consisting essentially of the nucleotide sequence of SEQ ID NO: 43 and (b) a nucleotide sequence encoding a therapeutic molecule.

### Additional Embodiments

The present disclosure also provides, but is not limited to, the embodiments described in the following numbered paragraphs.

1. A recombinant viral genome comprising an inhibitory nucleotide sequence of any one of SEQ ID NOS: 1-42.

2. The recombinant viral genome of paragraph 1 comprising the inhibitory nucleotide sequence of SEQ ID NO: 4.

3. The recombinant viral genome of paragraph 1 comprising the inhibitory nucleotide sequence of SEQ ID NO: 19.

4. The recombinant viral genome of paragraph 1 comprising the inhibitory nucleotide sequence of SEQ ID NO: 20.

5. The recombinant viral genome of paragraph 1 comprising the inhibitory nucleotide sequence of SEQ ID NO: 21.

6. The recombinant viral genome of paragraph 1 comprising the inhibitory nucleotide sequence of SEQ ID NO: 27.

7. The recombinant viral genome of paragraph 1 comprising the inhibitory nucleotide sequence of SEQ ID NO: 30.

8. The recombinant viral genome of paragraph 1 comprising the inhibitory nucleotide sequence of SEQ ID NO: 32.

9. The recombinant viral genome of paragraph 1 comprising the inhibitory nucleotide sequence of SEQ ID NO: 21 and the nucleotide sequence of SEQ ID NO: 30.

10. The recombinant viral genome of paragraph 1 comprising the inhibitory nucleotide sequence of SEQ ID NO: 40.

11. The recombinant viral genome of paragraph 1 comprising the inhibitory nucleotide sequence of SEQ ID NO: 41.

12. The recombinant viral genome of paragraph 1 comprising the inhibitory nucleotide sequence of SEQ ID NO: 42.

13. The recombinant viral genome of any one of paragraphs 1-12, wherein the recombinant viral genome is selected from the group consisting of adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, and human parvovirus B19.

14. The recombinant viral genome of paragraph 13, wherein the recombinant viral genome is an AAV genome.

15. The recombinant viral genome of any one of paragraphs 1-14, wherein the recombinant viral genome is single stranded.

16. The recombinant viral genome of any one of paragraphs 1-15, wherein the viral genome is packaged in a virion.

17. The recombinant viral genome of any one of paragraphs 1-16, wherein the recombinant viral genome comprises a therapeutic gene which is expressible in a human cell.

18. The recombinant viral genome of any one of paragraphs 1-17, wherein the recombinant viral genome is a cytotoxic virus for lysing target tumor cells.

19. The recombinant viral genome of any one of paragraphs 1-18, wherein the recombinant viral genome comprises a non-human gene.

20. The recombinant viral genome of any one of paragraphs 1-19, wherein the inhibitory nucleotide sequence is inserted downstream of or in a 3′ untranslated region of the viral genome.

21. The recombinant viral genome of any one of paragraphs 1-20, wherein the recombinant viral genome is covalently linked by a phosphodiester bond to the inhibitory nucleotide sequence.

22. The recombinant viral genome of any one of paragraphs 1-21 further comprising a detectable marker.

23. The recombinant viral genome of paragraph 22, wherein expression of the detectable marker is inducible.

24. The recombinant viral genome of any one of paragraphs 1-23, wherein the recombinant viral genome is self-complementary.

25. A method of treating a subject, comprising administering to the subject the recombinant viral genome of any one of paragraphs 1-23.

26. A method of producing a recombinant viral genome, the method comprising inserting into a viral genome the nucleotide sequence of any one of SEQ ID NOS: 1-42.

27. A nucleic acid vector comprising the nucleotide sequence of any one of SEQ ID NOS: 1-42.

28. A method comprising administering to a subject a recombinant viral genome comprising the inhibitory nucleotide sequence of SEQ ID NO: 21, the inhibitory nucleotide sequence of SEQ ID NO: 30, and a nucleotide sequence encoding a therapeutic molecule.

29. A method comprising administering to a subject a recombinant viral genome comprising the inhibitory nucleotide sequence of SEQ ID NO: 40 and a nucleotide sequence encoding a therapeutic molecule.

30. A method comprising administering to a subject a recombinant viral genome comprising (a) an inhibitory nucleotide sequence consisting essentially of the nucleotide sequence of SEQ ID NO: 42 and (b) a nucleotide sequence encoding a therapeutic molecule.

31. The method of any one of paragraphs 28-30, wherein NK-KB activation in the subject is reduced by at least 40%, at least 50%, at least 60%, at least 70%, at least 80, at least 90%, or at least 98%, relative to a control, optionally wherein the control is NK-κB activation in the subject administered a recombinant viral genome without an inhibitory nucleotide sequence.

### EXAMPLES

### Example 1

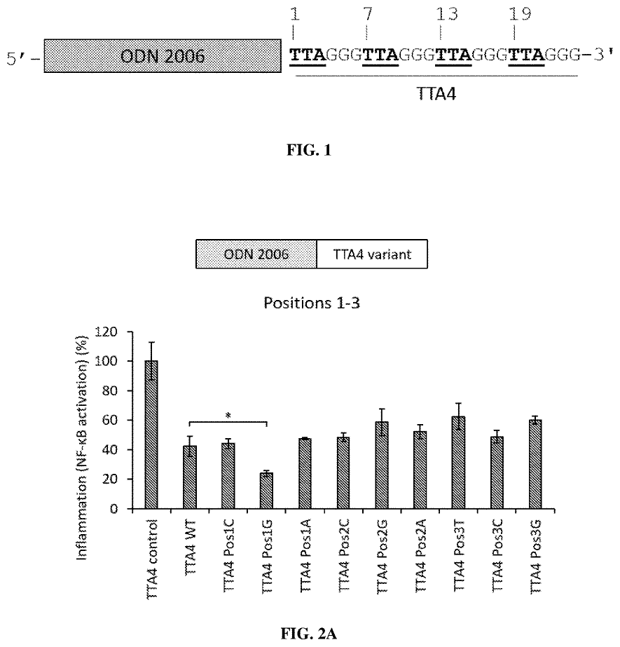

To test the efficacy of variants of TTA4 WT (a TLR9 inhibitory oligonucleotide comprising four tandem repeats of the telomeric motif TTAGGG) to block inflammation induced by the CpG DNA TLR9 ligand (ODN 2006), a library (Table 1) was designed. This library included single-stranded DNA oligonucleotide constructs comprising the inflammatory ODN 2006 sequence fused at the 3′ end to either the wild-type TTA4 sequence (TTA4 WT), a control sequence not expected to inhibit TLR9 (TTA4 control), or a TTA4 sequence containing a single nucleotide mutation (TTA4 Pos1C-TTA4 Pos21G) (FIG. 1). All oligonucleotides were synthesized with a phosphorothioate backbone for increased stability. A HEK293-based reporter cell line that constitutively expresses TLR9 to measure TLR9-mediated inflammation was used.

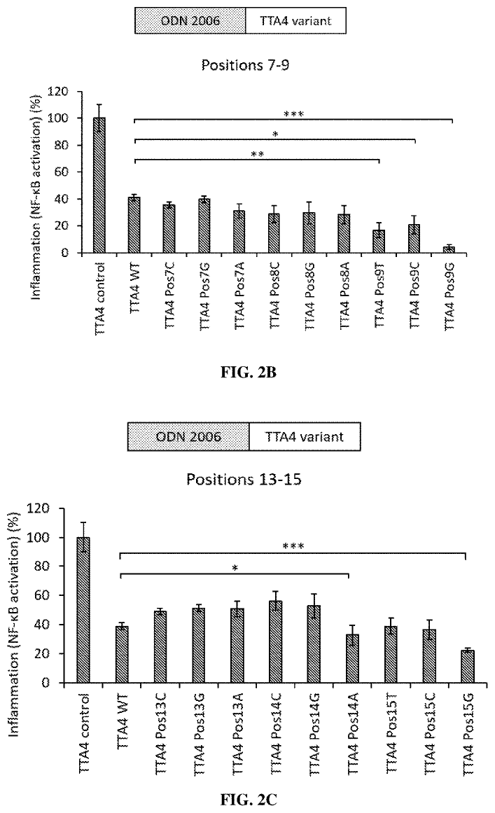

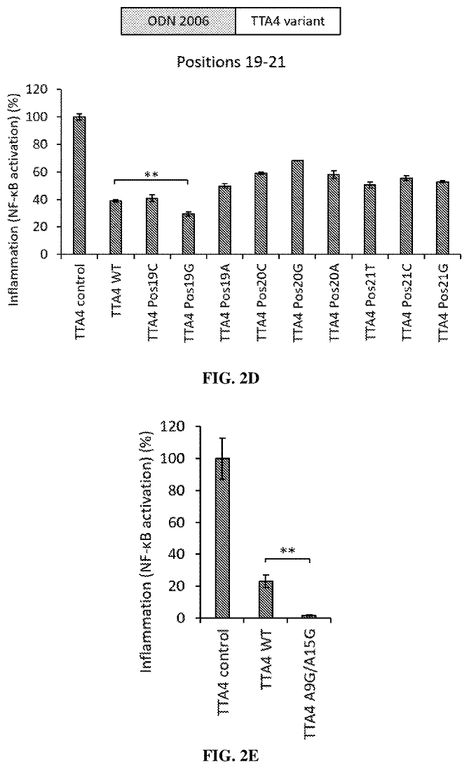

When oligonucleotides were applied at the concentration of 10 μM, the oligonucleotide containing the TTA4 control sequence induced robust inflammation. In contrast, the oligonucleotide containing TTA4 WT showed markedly reduced inflammation. Across the oligonucleotide library encoding single nucleotide changes to the TTA4 WT sequence, all TTA4 variant sequences tested retained their ability to reduce ODN 2006-mediated inflammation (FIGS. 2A-2D). Surprisingly, some TTA4 variant sequences demonstrated significantly more robust inhibitory activity compared to the TTA4 WT sequence. These included TTA4 Pos1C (SEQ ID NO: 4) (FIG. 2A), TTA4 Pos9T (SEQ ID NO: 19), TTA4 Pos9C (SEQ ID NO: 20), TTA4 Pos9G (SEQ ID NO: 21) (FIG. 2B), TTA4 Pos14A (SEQ ID NO: 27), TTA4 Pos15G (SEQ ID NO: 30) (FIG. 2C), and TTA4 Pos19G (SEQ ID NO: 32) (FIG. 2D). To test whether the mutations that most significantly improved TTA4 WT inhibition of ODN 2006-induced inflammation could work synergistically, a construct was made comprising ODN 2006 fused to a TTA4 sequence containing both the TTA4 Pos9G (SEQ ID NO: 21) and TTA4 Pos15G (SEQ ID NO: 30) mutations (TTA4 A9G/A15G) and was tested in the HEK293-TLR9 reporter system (FIG. 2E). Unexpectedly, TTA4 A9G/A15G demonstrated ˜98% inhibition of TLR9-mediated inflammation. Taken together, these data show that mutating the TTA4 sequence can give rise to variants with more potent TLR9 inhibitory activity than the original wild-type sequence. These novel TLR9 inhibitory sequences, in some instances, may be used for suppressing TLR9 activation in the context of a longer piece of inflammatory DNA, such as a viral vector.

Materials and Methods: HEK-Blue-hTLR9 reporter cells were incubated with ODN 2006 fused to each of the TTA4 variant sequences indicated on the x-axis (single-stranded DNA with a phosphorothioate backbone) at a concentration of 10 μM for 18 hours. 50 μl cell supernatant was then incubated with 150 μl HEK-BLUE Detection for 4-6 hours at 37° C., and absorbance at 630 nm was measured by a plate reader Inflammation was defined as the amount of NF-kB activity induced above mock-treated cells, and this was set to 100% for cells treated with TTA4 control.

TABLE 1
Sequence Library (mutations underlined)

| Name | Sequence | SEQ ID NO: |
|---|---|---|
| ODN 2006 | TCGTCGTTTTGTCGTTTTGTCGTT | 1 |
| TTA4 WT | TTAGGGTTAGGGTTAGGGTTAGGG | 2 |
| TTA4 control | TTATTATTATTATTATTATTATTA | 3 |
| TTA4 Pos1C | [symbol] TAGGGTTAGGGTTAGGGTTAGGG | 4 |
| TTA4 Pos1G | [symbol] TAGGGTTAGGGTTAGGGTTAGGG | 5 |
| TTA4 Pos1A | [symbol] TAGGGTTAGGGTTAGGGTTAGGG | 6 |
| TTA4 Pos2C | TCAGGGTTAGGGTTAGGGTTAGGG | 7 |
| TTA4 Pos2G | TGAGGGTTAGGGTTAGGGTTAGGG | 8 |
| TTA4 Pos2A | TAAGGGTTAGGGTTAGGGTTAGGG | 9 |
| TTA4 Pos3T | TT [symbol] GGGTTAGGGTTAGGGTTAGGG | 10 |
| TTA4 Pos3C | TT [symbol] GGGTTAGGGTTAGGGTTAGGG | 11 |
| TTA4 Pos3G | TTGGGGTTAGGGTTAGGGTTAGGG | 12 |
| TTA4 Pos7C | TTAGGGCTAGGGTTAGGGTTAGGG | 13 |
| TTA4 Pos7G | TTAGGGGTAGGGTTAGGGTTAGGG | 14 |
| TTA4 Pos7A | TTAGGG [symbol] TAGGGTTAGGGTTAGGG | 15 |
| TTA4 Pos8C | TTAGGGTCAGGGTTAGGGTTAGGG | 16 |
| TTA4 Pos8G | TTAGGGTGAGGGTTAGGGTTAGGG | 17 |
| TTA4 Pos8A | TTAGGGTAAGGGTTAGGGTTAGGG | 18 |
| TTA4 Pos9T | TTAGGGTT [symbol] GGGTTAGGGTTAGGG | 19 |
| TTA4 Pos9C | TTAGGGTTCGGGTTAGGGTTAGGG | 20 |
| TTA4 Pos9G | TTAGGGTTGGGGTTAGGGTTAGGG | 21 |
| TTA4 Pos13C | TTAGGGTTAGGGCTAGGGTTAGGG | 22 |
| TTA4 Pos13G | TTAGGGTTAGGGGTAGGGTTAGGG | 23 |
| TTA4 Pos13A | TTAGGGTTAGGGATAGGGTTAGGG | 24 |
| TTA4 Pos14C | TTAGGGTTAGGGTCAGGGTTAGGG | 25 |
| TTA4 Pos14G | TTAGGGTTAGGGTGAGGGTTAGGG | 26 |
| TTA4 Pos14A | TTAGGGTTAGGGTAAGGGTTAGGG | 27 |
| TTA4 Pos15T | TTAGGGTTAGGGTTTGGGTTAGGG | 28 |
| TTA4 Pos15C | TTAGGGTTAGGGTTCGGGTTAGGG | 29 |
| TTA4 Pos15G | TTAGGGTTAGGGTTGGGGTTAGGG | 30 |
| TTA4 Pos19C | TTAGGGTTAGGGTTAGGGCTAGGG | 31 |
| TTA4 Pos19G | TTAGGGTTAGGGTTAGGGGTAGGG | 32 |
| TTA4 Pos19A | TTAGGGTTAGGGTTAGGGATAGGG | 33 |
| TTA4 Pos20C | TTAGGGTTAGGGTTAGGGTCAGGG | 34 |
| TTA4 Pos20G | TTAGGGTTAGGGTTAGGGT [symbol] AGGG | 35 |
| TTA4 Pos20A | TTAGGGTTAGGGTTAGGGTAAGGG | 36 |
| TTA4 Pos21T | TTAGGGTTAGGGTTAGGGTTTGGG | 37 |
| TTA4 Pos21C | TTAGGGTTAGGGTTAGGGTTCGGG | 38 |
| TTA4 Pos21G | TTAGGGTTAGGGTTAGGGTTGGGG | 39 |
| TTA4 A9G/A15G | TTAGGGTTGGGGTTGGGGTTAGGG | 40 |
| TTA3 WT | TTAGGGTTAGGGTTAGGG | 41 |
| TTA2 WT | TTAGGGTTAGGG | 42 |
| TTA1 WT | TTAGGG | 43 |
| TTA3 control | TTATTATTATTATTATTA | 44 |
| TTA2 control | TTATTATTATTA | 45 |
| TTA1 control | TTATTA | 46 |

### Example 2

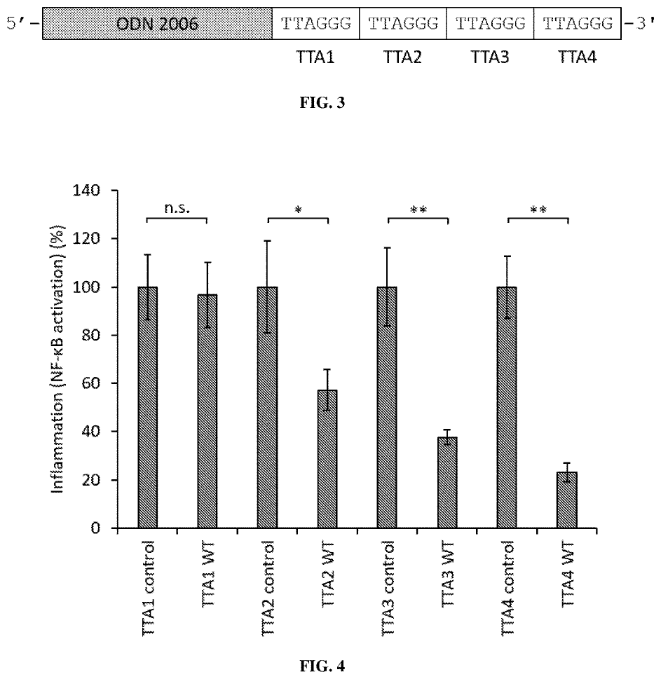

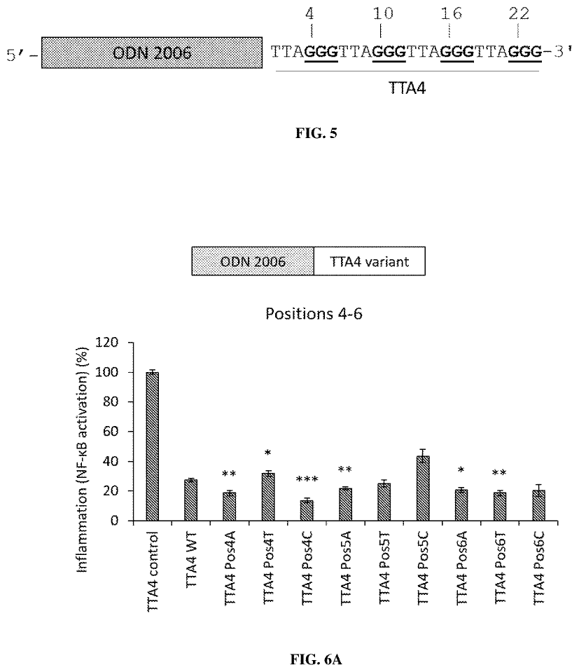

The TLR9 inhibitory telomeric sequence TTA4 WT has been characterized as a potent inhibitor of TLR9-mediated inflammation, and it is composed of four tandem repeats of a telomeric TTAGGG motif. When supplied in trans, two, three, and four tandem copies of the TTAGGG motif have been shown to inhibit CpG DNA-induced immune activation (Gursel, I et al. J Immunol. 2003;171(3):1393-400). However, in the context of a longer piece of DNA (i.e. when fused to an inflammatory DNA sequence), it is unknown whether the number of TTAGGG repeats administered in cis (fused to the 3′ end of an inflammatory oligonucleotide sequence) affects the ability of the TTAGGG motif to inhibit TLR9 activation. To determine the minimal number of tandem repeats of the TTAGGG motif required to inhibit TLR9, constructs were designed comprising the inflammatory ODN 2006 sequence fused to either one (TTA1 WT), two (TTA2 WT), three (TTA3 WT), or four (TTA4 WT) copies of the TTAGGG motif (FIG. 3). All oligonucleotides were synthesized with a phosphorothioate backbone for increased stability. A HEK293-based reporter cell line that constitutively expresses TLR9 to measure TLR9-mediated inflammation was used. When oligonucleotides were applied at the concentration of 10 μM, the oligonucleotide containing the TTA1 control sequence induced robust inflammation, and the oligonucleotide containing the TTA1 WT sequence induced comparable levels of inflammation, suggesting that a single copy of the TTAGGG motif does not confer TLR9 inhibitory activity (FIG. 4). The oligonucleotide containing the TTA4 WT sequence showed robust inhibition of inflammation compared to the oligonucleotide containing the TTA4 control sequence. Surprisingly, oligonucleotides containing two (TTA2 WT; SEQ ID NO: 42) and three (TTA3 WT; SEQ ID NO: 41) copies of the TTAGGG motif both showed significant suppressive activity compared to oligonucleotides containing the control sequences. These data suggest that two tandem repeats of the TTAGGG (SEQ ID NO: 43) motif is the minimal sequence required to retain TLR9 inhibitory activity.

Materials and Methods: HEK-Blue-hTLR9 reporter cells were incubated with ODN 2006 fused to one, two, three, or four copies of the TTAGGG motif, as indicated on the x-axis (single-stranded DNA with a phosphorothioate backbone) at a concentration of 10 μM for 18 hours (FIGS. 2A-2E). 50 μl cell supernatant was then incubated with 150 μl HEK-BLUE Detection for 4-6 hours at 37° C., and absorbance at 630 nm was measured by a plate reader. Inflammation was defined as the amount of NF-kB activity induced above mock-treated cells, and this was set to 100% for cells treated with respective TTA control (e.g., TTA1 control, TTA2 control etc.).

### HEK293-TLR9 Reporter Cell Assay

A HEK293-based reporter cell line stably expressing human TLR9 and an inducible SEAP (secreted embryonic alkaline phosphatase) reporter gene was obtained (HEK-Blue-hTLR9, Invivogen). The SEAP gene is under the control of the IFN-β minimal promoter fused to five NF-κB and AP-1-binding sites. Stimulation with a TLR9 ligand activated NF-κB and AP-1 which induced the production of SEAP. Levels of SEAP were measured using HEK-Blue Detection to determine the amount of inflammation. Cells were cultured in Dulbeccco's Modified Eagle's Medium (DMEM) supplemented with 10% (v/v) heat-inactivated fetal bovine serum and 1% (v/v) penicillin-streptomycin (Invitrogen) under standard tissue culture conditions. All single-stranded DNA oligonucleotides were synthesized with a phosphorothioate backbone for increased stability (IDT). One set of oligonucleotides used in the present disclosure was composed of variants of a base construct comprising the TLR9 ligand sequence ODN 2006 fused to the TLR9 inhibitory sequence TTA4 or the corresponding control sequence of the same length. Another set of oligonucleotides used in the present disclosure was composed of the ODN 2006 sequence fused to one, two, three, or four copies of the TTAGGG motif, or the corresponding control sequences of the same length. Oligonucleotides were incubated at a concentration of 10 μM with 6×10⁴ HEK-Blue-hTLR9 cells in 200 μl of DMEM growth media per well in 96-well flat bottom plates for 18 hours, and 50 μl media was aspirated and incubated with 150 μl HEK-Blue Detection media (Invivogen) for 3-6 hours at 37° C. and absorbance was read at 630 nm on a plate reader (BioTek).

### Example 3

Further In vitro studies were conducted to show the effect of mutating the specific positions of the TLR9 inhibitory oligonucleotide TTA4 on inflammation induced by the TLR9 activating sequence ODN 2006 (see FIG. 5).

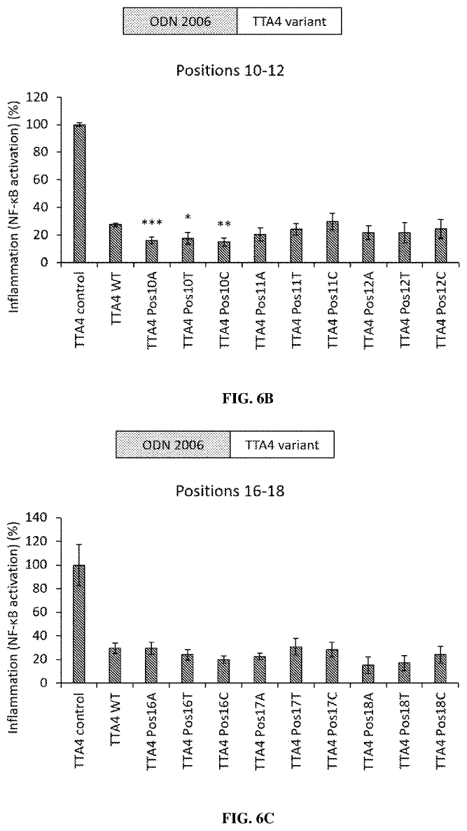

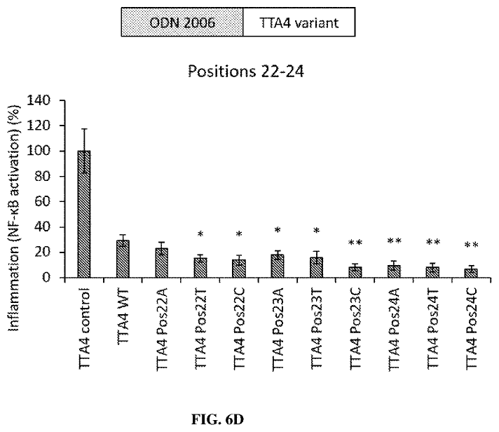

Across the oligonucleotide library encoding single nucleotide changes to the TTA4 WT sequence in the GGG regions, all TTA4 variant sequences tested retained their ability to inhibit ODN 2006-mediated inflammation (FIGS. 6A-6D). Surprisingly, some TTA4 variant sequences demonstrated significantly more robust inhibitory activity compared to the TTA4 WT sequence. These included TTA4 Pos4A (SEQ ID NO: 47), TTA4 Pos4T (SEQ ID NO: 48), TTA4 Pos4C (SEQ ID NO: 49), TTA4 Pos6A (SEQ ID NO: 53), and TTA4 Pos6T (SEQ ID NO: 54) (FIG. 6A); TTA4 Pos10A (SEQ ID NO: 56), TTA4 PoslOT (SEQ ID NO: 57), and TTA4 PoslOC (SEQ ID NO: 58) (FIG. 6B); TTA4 Pos22T (SEQ ID NO: 75), TTA4 Pos22C (SEQ ID NO: 76), TTA4 Pos23A (SEQ ID NO: 77), TTA4 Pos23T (SEQ ID NO: 78), TTA4 Pos23C (SEQ ID NO: 79), TTA4 Pos24A (SEQ ID NO: 80), TTA4 Pos24T (SEQ ID NO: 81), and TTA4 Pos24C (SEQ ID NO: 82) (FIG. 6D).

Materials and Methods: HEK-Blue-hTLR9 reporter cells were incubated with ODN 2006 fused to each of the TTA4 variant sequences indicated on the x-axis (single-stranded DNA with a phosphorothioate backbone) of at a concentration of 10 μM for 18 h (FIGS. 6A-6D). 50 μl cell supernatant was then incubated with 150 μl HEK-BLUE Detection for 4-6 h at 37 ° C., and absorbance at 630 nm was measured by a plate reader.

TABLE 2
Sequence Library (mutations underlined)

| Name | Sequence | SEQ ID NO: |
|---|---|---|
| ODN 2006 | TCGTCGTTTTGTCGTTTTGTCGTT | 1 |
| TTA4 WT | TTAGGGTTAGGGTTAGGGTTAGGG | 2 |
| TTA4 Pos4A | TTA [symbol] GGTTAGGGTTAGGGTTAGGG | 47 |
| TTA4 Pos4T | TT [symbol] TGGTTAGGGTTAGGGTTAGGG | 48 |
| TTA4 Pos4C | TTA [symbol] GGTTAGGGTTAGGGTTAGGG | 49 |
| TTA4 Pos5A | TTAG [symbol] GTTAGGGTTAGGGTTAGGG | 50 |
| TTA4 Pos5T | TTAG [symbol] GTTAGGGTTAGGGTTAGGG | 51 |
| TTA4 Pos5C | TTAGCGTTAGGGTTAGGGTTAGGG | 52 |
| TTA4 Pos6A | TTAGGATTAGGGTTAGGGTTAGGG | 53 |
| TTA4 Pos6T | TTAGGTTTAGGGTTAGGGTTAGGG | 54 |
| TTA4 Pos6C | TTAGGCTTAGGGTTAGGGTTAGGG | 55 |
| TTA4 Pos10A | TTAGGGTTAAGGTTAGGGTTAGGG | 56 |
| TTA4 Pos10T | TTAGGGTTATGGTTAGGGTTAGGG | 57 |
| TTA4 Pos10C | TTAGGGTTACGGTTAGGGTTAGGG | 58 |
| TTA4 Pos11 A | TTAGGGTTAGAGTTAGGGTTAGGG | 59 |
| TTA4 Pos11T | TTAGGGTTAGTGTTAGGGTTAGGG | 60 |
| TTA4 Pos11C | TTAGGGTTAGCGTTAGGGTTAGGG | 61 |
| TTA4 Pos12A | TTAGGGTTAGGATTAGGGTTAGGG | 62 |
| TTA4 Pos12T | TTAGGGTTAGGTTTAGGGTTAGGG | 63 |
| TTA4 Pos12C | TTAGGGTTAGGCTTAGGGTTAGGG | 64 |
| TTA4 Pos16A | TTAGGGTTAGGGTTAAGGTTAGGG | 65 |
| TTA4 Pos16T | TTAGGGTTAGGGTTATGGTTAGGG | 66 |
| TTA4 Pos16C | TTAGGGTTAGGGTTACGGTTAGGG | 67 |
| TTA4 Pos17A | TTAGGGTTAGGGTTAGAGTTAGGG | 68 |
| TTA4 Pos17T | TTAGGGTTAGGGTTAGTGTTAGGG | 69 |
| TTA4 Pos17C | TTAGGGTTAGGGTTAGCGTTAGGG | 70 |
| TTA4 Pos18A | TTAGGGTTAGGGTTAGGATTAGGG | 71 |
| TTA4 Pos18T | TTAGGGTTAGGGTTAGGTTTAGGG | 72 |
| TTA4 Pos18C | TTAGGGTTAGGGTTAGGCTTAGGG | 73 |
| TTA4 Pos22A | TTAGGGTTAGGGTTAGGGTTAAGG | 74 |
| TTA4 Pos22T | TTAGGGTTAGGGTTAGGGTTATGG | 75 |
| TTA4 Pos22C | TTAGGGTTAGGGTTAGGGTTACGG | 76 |
| TTA4 Pos23A | TTAGGGTTAGGGTTAGGGTTAGAG | 77 |
| TTA4 Pos23T | TTAGGGTTAGGGTTAGGGTTAGTG | 78 |
| TTA4 Pos23C | TTAGGGTTAGGGTTAGGGTTAGCG | 79 |
| TTA4 Pos24A | TTAGGGTTAGGGTTAGGGTTAGGA | 80 |
| TTA4 Pos24T | TTAGGGTTAGGGTTAGGGTTAGGT | 81 |
| TTA4 Pos24C | TTAGGGTTAGGGTTAGGGTTAGGC | 82 |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A nucleic acid vector comprising:
(a) an inhibitory oligonucleotide variant comprising an inhibitory nucleotide sequence that comprises a nucleotide substitution at one or more of positions 1, 2, 3, 7, 8, 9, 13, 14, 15, 19, 20, and 21, relative to the nucleotide sequence of SEQ ID NO: 2; and
(b) a therapeutic nucleotide sequence.

2. The nucleic acid vector of claim 1, wherein the inhibitory nucleotide sequence has at least 90% identity to the nucleotide sequence of SEQ ID NO: 2.

3. The nucleic acid vector of claim 1, wherein the inhibitory nucleotide sequence comprises a nucleotide substitution at position 9 and at position 15, relative to the nucleotide sequence of SEQ ID NO: 2.

4. The nucleic acid vector of claim 3, wherein the inhibitory nucleotide sequence comprises a G at position 9 and a G at position 15, relative to the nucleotide sequence of SEQ ID NO: 2.

5. The nucleic acid vector of claim 4, wherein the inhibitory nucleotide sequence comprises a nucleotide sequence having at least 95% identity to the nucleotide sequence of SEQ ID NO: 40.

6. The nucleic acid vector of claim 5, wherein the inhibitory nucleotide sequence comprises the nucleotide sequence of SEQ ID NO: 40.

7. The nucleic acid vector of any one of claims 1-4, wherein the inhibitory nucleotide sequence comprises a nucleotide substitution at position 1, relative to the nucleotide sequence of SEQ ID NO: 2.

8. The nucleic acid vector of claim 7, wherein the inhibitory nucleotide sequence comprises a cytosine (C) at position 1, relative to the nucleotide sequence of SEQ ID NO: 2.

9. The nucleic acid vector of claim 8, wherein the inhibitory nucleotide sequence comprises a nucleotide sequence having at least 95% identity to the nucleotide sequence of SEQ ID NO: 4.

10. The nucleic acid vector of claim 9, wherein the inhibitory nucleotide sequence comprises the nucleotide sequence of SEQ ID NO: 4.

11. The nucleic acid vector of any one of claims 1-4, 7, and 8, wherein the inhibitory nucleotide sequence comprises a nucleotide substitution at position 9, relative to the nucleotide sequence of SEQ ID NO: 2.

12. The nucleic acid vector of claim 11, wherein the inhibitory nucleotide sequence comprises a thymidine (T) at position 9, relative to the nucleotide sequence of SEQ ID NO: 2.

13. The nucleic acid vector of claim 12, wherein the inhibitory nucleotide sequence comprises a nucleotide sequence having at least 95% identity to the nucleotide sequence of SEQ ID NO: 19.

14. The nucleic acid vector of claim 13, wherein the inhibitory nucleotide sequence comprises the nucleotide sequence of SEQ ID NO: 19.

15. The nucleic acid vector of any one of claims 1-4, 7, 8, and 11, wherein the inhibitory nucleotide sequence comprises a cytosine (C) at position 9, relative to the nucleotide sequence of SEQ ID NO: 2.

16. The nucleic acid vector of claim 15, wherein the inhibitory nucleotide sequence comprises a nucleotide sequence having at least 95% identity to the nucleotide sequence of SEQ ID NO: 20.

17. The nucleic acid vector of claim 16, wherein the inhibitory nucleotide sequence comprises the nucleotide sequence of SEQ ID NO: 20.

18. The nucleic acid vector of any one of claims 1-4, 7, 8, and 11, wherein the inhibitory nucleotide sequence comprises a guanosine (G) at position 9, relative to the nucleotide sequence of SEQ ID NO: 2.

19. The nucleic acid vector of claim 18, wherein the inhibitory nucleotide sequence comprises a nucleotide sequence having at least 95% identity to the nucleotide sequence of SEQ ID NO: 21.

20. The nucleic acid vector of claim 19, wherein the inhibitory nucleotide sequence comprises the nucleotide sequence of SEQ ID NO: 21.

21. The nucleic acid vector of any one of claims 1-4, 7, 8, 11, 12, 15, and 18, wherein the inhibitory nucleotide sequence comprises a nucleotide substitution at position 14, relative to the nucleotide sequence of SEQ ID NO: 2.

22. The nucleic acid vector of claim 21, wherein the inhibitory nucleotide sequence comprises an adenosine (A) at position 14, relative to the nucleotide sequence of SEQ ID NO: 2.

23. The nucleic acid vector of claim 22, wherein the inhibitory nucleotide sequence comprises a nucleotide sequence having at least 95% identity to the nucleotide sequence of SEQ ID NO: 27.

24. The nucleic acid vector of claim 23, wherein the inhibitory nucleotide sequence comprises the nucleotide sequence of SEQ ID NO: 27.

25. The nucleic acid vector of any one of claims 1-4, 7, 8, 11, 12, 15, 18, 21, and 22, wherein the inhibitory nucleotide sequence comprises a nucleotide substitution at position 15, relative to the nucleotide sequence of SEQ ID NO: 2.

26. The nucleic acid vector of claim 25, wherein the inhibitory nucleotide sequence comprises a G at position 15, relative to the nucleotide sequence of SEQ ID NO: 2.

27. The nucleic acid vector of claim 26, wherein the inhibitory nucleotide sequence comprises a nucleotide sequence having at least 95% identity to the nucleotide sequence of SEQ ID NO: 30.

28. The nucleic acid vector of claim 27, wherein the inhibitory nucleotide sequence comprises the nucleotide sequence of SEQ ID NO: 30.

29. The nucleic acid vector of any one of claims 1-4, 7, 8, 11, 12, 15, 18, 21, 22, 25, and 26, wherein the inhibitory nucleotide sequence comprises a nucleotide substitution at position 19, relative to the nucleotide sequence of SEQ ID NO: 2.

30. The nucleic acid vector of claim 29, wherein the inhibitory nucleotide sequence comprises a G at position 19, relative to the nucleotide sequence of SEQ ID NO: 2.

31. The nucleic acid vector of claim 30, wherein the inhibitory nucleotide sequence comprises a nucleotide sequence having at least 95% identity to the nucleotide sequence of SEQ ID NO: 32.

32. The nucleic acid vector of claim 31, wherein the inhibitory nucleotide sequence comprises the nucleotide sequence of SEQ ID NO: 32.

33. The nucleic acid vector of any one of claims 1-4, 7, 8, 11, 12, 15, 18, 21, 22, 25, 26, 29 and 30 further comprising an inhibitory oligonucleotide variant comprising an inhibitory nucleotide sequence having a G at position 15, relative to the nucleotide sequence of SEQ ID NO: 2.

34. The nucleic acid vector of claim 33, wherein the inhibitory nucleotide sequence comprises the nucleotide sequence of SEQ ID NO: 30.

35. A nucleic acid vector comprising:
(a) an inhibitory oligonucleotide variant comprising an inhibitory nucleotide sequence that comprises a nucleotide substitution at one or more of positions 4, 5, 6, 10, 11, 12, 16, 17, 18, 22, 23, and 24, relative to the nucleotide sequence of SEQ ID NO: 2; and
(b) a therapeutic nucleotide sequence.

36. The nucleic acid vector of claim 35, wherein the inhibitory nucleotide sequence has at least 90% identity to the nucleotide sequence of SEQ ID NO: 2.

37. The nucleic acid vector of claim 35, wherein the inhibitory nucleotide sequence comprises a nucleotide substitution at position 4, relative to the nucleotide sequence of SEQ ID NO: 2.

38. The nucleic acid vector of claim 37, wherein the inhibitory nucleotide sequence comprises an adenosine (A) at position 4, relative to the nucleotide sequence of SEQ ID NO: 2.

39. The nucleic acid vector of claim 38, wherein the inhibitory nucleotide sequence comprises a nucleotide sequence having at least 95% identity to the nucleotide sequence of SEQ ID NO: 47.

40. The nucleic acid vector of claim 39, wherein the inhibitory nucleotide sequence comprises the nucleotide sequence of SEQ ID NO: 47.

41. The nucleic acid vector of any one of claims 35-37, wherein the inhibitory nucleotide sequence comprises a thymidine (T) at position 4, relative to the nucleotide sequence of SEQ ID NO: 2.

42. The nucleic acid vector of claim 41, wherein the inhibitory nucleotide sequence comprises a nucleotide sequence having at least 95% identity to the nucleotide sequence of SEQ ID NO: 48.

43. The nucleic acid vector of claim 42, wherein the inhibitory nucleotide sequence comprises the nucleotide sequence of SEQ ID NO: 48.

44. The nucleic acid vector of any one of claims 35-37, wherein the inhibitory nucleotide sequence comprises a cytosine (C) at position 4, relative to the nucleotide sequence of SEQ ID NO: 2.

45. The nucleic acid vector of claim 44, wherein the inhibitory nucleotide sequence comprises a nucleotide sequence having at least 95% identity to the nucleotide sequence of SEQ ID NO: 49.

46. The nucleic acid vector of claim 45, wherein the inhibitory nucleotide sequence comprises the nucleotide sequence of SEQ ID NO: 49.

47. The nucleic acid vector of any one of claims 35-38, 41 and 44, wherein the inhibitory nucleotide sequence comprises a nucleotide substitution at position 6, relative to the nucleotide sequence of SEQ ID NO: 2.

48. The nucleic acid vector of claim 47, wherein the inhibitory nucleotide sequence comprises an A at position 6, relative to the nucleotide sequence of SEQ ID NO: 2.

49. The nucleic acid vector of claim 48, wherein the inhibitory nucleotide sequence comprises a nucleotide sequence having at least 95% identity to the nucleotide sequence of SEQ ID NO: 53.

50. The nucleic acid vector of claim 49, wherein the inhibitory nucleotide sequence comprises the nucleotide sequence of SEQ ID NO: 53.

51. The nucleic acid vector of any one of claims 35-38, 41, 44, and 47, wherein the inhibitory nucleotide sequence comprises a T at position 6, relative to the nucleotide sequence of SEQ ID NO: 2.

52. The nucleic acid vector of claim 51, wherein the inhibitory nucleotide sequence comprises a nucleotide sequence having at least 95% identity to the nucleotide sequence of SEQ ID NO: 54.

53. The nucleic acid vector of claim 52, wherein the inhibitory nucleotide sequence comprises the nucleotide sequence of SEQ ID NO: 54.

54. The nucleic acid vector of any one of claims 35-38, 41, 44, 47, 48, and 51, wherein the inhibitory nucleotide sequence comprises a nucleotide substitution at position 10, relative to the nucleotide sequence of SEQ ID NO: 2.

55. The nucleic acid vector of claim 54, wherein the inhibitory nucleotide sequence comprises an A at position 10, relative to the nucleotide sequence of SEQ ID NO: 2.

56. The nucleic acid vector of claim 55, wherein the inhibitory nucleotide sequence comprises a nucleotide sequence having at least 95% identity to the nucleotide sequence of SEQ ID NO: 56.

57. The nucleic acid vector of claim 56, wherein the inhibitory nucleotide sequence comprises the nucleotide sequence of SEQ ID NO: 56.

58. The nucleic acid vector of any one of claims 35-38, 41, 44, 47, 48, 51, and 54, wherein the inhibitory nucleotide sequence comprises a T at position 10, relative to the nucleotide sequence of SEQ ID NO: 2.

59. The nucleic acid vector of claim 58, wherein the inhibitory nucleotide sequence comprises a nucleotide sequence having at least 95% identity to the nucleotide sequence of SEQ ID NO: 57.

60. The nucleic acid vector of claim 59, wherein the inhibitory nucleotide sequence comprises the nucleotide sequence of SEQ ID NO: 57.

61. The nucleic acid vector of any one of claims 35-38, 41, 44, 47, 48, 51, and 54, wherein the inhibitory nucleotide sequence comprises a C at position 10, relative to the nucleotide sequence of SEQ ID NO: 2.

62. The nucleic acid vector of claim 61, wherein the inhibitory nucleotide sequence comprises a nucleotide sequence having at least 95% identity to the nucleotide sequence of SEQ ID NO: 58.

63. The nucleic acid vector of claim 62, wherein the inhibitory nucleotide sequence comprises the nucleotide sequence of SEQ ID NO: 58.

64. The nucleic acid vector of any one of claims 35-38, 41, 44, 47, 48, 51, 54, 55, 58, and 61, wherein the inhibitory nucleotide sequence comprises a nucleotide substitution at position 22, relative to the nucleotide sequence of SEQ ID NO: 2.

65. The nucleic acid vector of claim 64, wherein the inhibitory nucleotide sequence comprises a T at position 22, relative to the nucleotide sequence of SEQ ID NO: 2.

66. The nucleic acid vector of claim 65, wherein the inhibitory nucleotide sequence comprises a nucleotide sequence having at least 95% identity to the nucleotide sequence of SEQ ID NO: 75.

67. The nucleic acid vector of claim 66, wherein the inhibitory nucleotide sequence comprises the nucleotide sequence of SEQ ID NO: 75.

68. The nucleic acid vector of any one of claims 35-38, 41, 44, 47, 48, 51, 54, 55, 58, 61, and 64, wherein the inhibitory nucleotide sequence comprises a C at position 22, relative to the nucleotide sequence of SEQ ID NO: 2.

69. The nucleic acid vector of claim 68, wherein the inhibitory nucleotide sequence comprises a nucleotide sequence having at least 95% identity to the nucleotide sequence of SEQ ID NO: 76.

70. The nucleic acid vector of claim 69, wherein the inhibitory nucleotide sequence comprises the nucleotide sequence of SEQ ID NO: 76.

71. The nucleic acid vector of any one of claims 35-38, 41, 44, 47, 48, 51, 54, 55, 58, 61, 64, 65, and 68, wherein the inhibitory nucleotide sequence comprises a nucleotide substitution at position 23, relative to the nucleotide sequence of SEQ ID NO: 2.

72. The nucleic acid vector of claim 71, wherein the inhibitory nucleotide sequence comprises an A at position 23, relative to the nucleotide sequence of SEQ ID NO: 2.

73. The nucleic acid vector of claim 72, wherein the inhibitory nucleotide sequence comprises a nucleotide sequence having at least 95% identity to the nucleotide sequence of SEQ ID NO: 77.

74. The nucleic acid vector of claim 73, wherein the inhibitory nucleotide sequence comprises the nucleotide sequence of SEQ ID NO: 77.

75. The nucleic acid vector of any one of claims 35-38, 41, 44, 47, 48, 51, 54, 55, 58, 61, 64, 65, 68, and 71, wherein the inhibitory nucleotide sequence comprises a T at position 23, relative to the nucleotide sequence of SEQ ID NO: 2.

76. The nucleic acid vector of claim 75, wherein the inhibitory nucleotide sequence comprises a nucleotide sequence having at least 95% identity to the nucleotide sequence of SEQ ID NO: 78.

77. The nucleic acid vector of claim 76, wherein the inhibitory nucleotide sequence comprises the nucleotide sequence of SEQ ID NO: 78.

78. The nucleic acid vector of any one of claims 35-38, 41, 44, 47, 48, 51, 54, 55, 58, 61, 64, 65, 68, and 71, wherein the inhibitory nucleotide sequence comprises a C at position 23, relative to the nucleotide sequence of SEQ ID NO: 2.

79. The nucleic acid vector of claim 78, wherein the inhibitory nucleotide sequence comprises a nucleotide sequence having at least 95% identity to the nucleotide sequence of SEQ ID NO: 79.

80. The nucleic acid vector of claim 79, wherein the inhibitory nucleotide sequence comprises the nucleotide sequence of SEQ ID NO: 79.

81. The nucleic acid vector of any one of claims 35-38, 41, 44, 47, 48, 51, 54, 55, 58, 61, 64, 65, 68, 71, 72, 75, and 78, wherein the inhibitory nucleotide sequence comprises a nucleotide substitution at position 24, relative to the nucleotide sequence of SEQ ID NO: 2.

82. The nucleic acid vector of claim 81, wherein the inhibitory nucleotide sequence comprises an A at position 24, relative to the nucleotide sequence of SEQ ID NO: 2.

83. The nucleic acid vector of claim 82, wherein the inhibitory nucleotide sequence comprises a nucleotide sequence having at least 95% identity to the nucleotide sequence of SEQ ID NO: 80.

84. The nucleic acid vector of claim 83, wherein the inhibitory nucleotide sequence comprises the nucleotide sequence of SEQ ID NO: 80.

85. The nucleic acid vector of any one of claims 35-38, 41, 44, 47, 48, 51, 54, 55, 58, 61, 64, 65, 68, 71, 72, 75, 78, and 81, wherein the inhibitory nucleotide sequence comprises a T at position 24, relative to the nucleotide sequence of SEQ ID NO: 2.

86. The nucleic acid vector of claim 85, wherein the inhibitory nucleotide sequence comprises a nucleotide sequence having at least 95% identity to the nucleotide sequence of SEQ ID NO: 81.

87. The nucleic acid vector of claim 86, wherein the inhibitory nucleotide sequence comprises the nucleotide sequence of SEQ ID NO: 81.

88. The nucleic acid vector of any one of claims 35-38, 41, 44, 47, 48, 51, 54, 55, 58, 61, 64, 65, 68, 71, 72, 75, 78, and 81, wherein the inhibitory nucleotide sequence comprises a C at position 24, relative to the nucleotide sequence of SEQ ID NO: 2.

89. The nucleic acid vector of claim 88, wherein the inhibitory nucleotide sequence comprises a nucleotide sequence having at least 95% identity to the nucleotide sequence of SEQ ID NO: 82.

90. The nucleic acid vector of claim 89, wherein the inhibitory nucleotide sequence comprises the nucleotide sequence of SEQ ID NO: 82.

91. A nucleic acid vector comprising:
(a) an inhibitory oligonucleotide consisting of SEQ ID NO: 41 or 42; and
(b) a therapeutic nucleotide sequence.

92. The nucleic acid vector of any one of claims 1-91, wherein the nucleic acid vector is a recombinant viral genome.

93. The nucleic acid vector of claim 92, wherein the recombinant viral genome is selected from the group consisting of adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, and human parvovirus B19.

94. The nucleic acid vector of claim 93, wherein the recombinant viral genome is an AAV genome.

95. The nucleic acid vector of any one of claims 1-94, wherein the recombinant viral genome is single stranded.

96. The nucleic acid vector of any one of claims 1-95, wherein the recombinant viral genome is self-complementary.

97. The nucleic acid vector of any one of claims 92-96, wherein the viral genome is packaged in a virion.

98. The nucleic acid vector of any one of claims 92-97, wherein the inhibitory nucleotide sequence is inserted downstream of or in a 3′ untranslated region of the viral genome.

99. The nucleic acid vector of any one of claims 92-98, wherein the recombinant viral genome is covalently linked by a phosphodiester bond to the inhibitory nucleotide sequence.

100. The nucleic acid vector of any one of claims 92-99 further comprising a detectable marker.

101. The nucleic acid vector of claim 100, wherein expression of the detectable marker is inducible.

102. The nucleic acid vector of any one of claims 92-101, wherein the therapeutic nucleotide sequence is a therapeutic DNA, a therapeutic RNA, or encodes a therapeutic protein.

103. A method of treating a subject, comprising administering to the subject the nucleic acid vector of any one of claims 92-102.

104. The method of claim 103, wherein the nucleic acid vector is administered by intravenous, intramuscular, subretinal, intravitreal, intrathecal, intraparenchymal, and intracranial injection.


SEQUENCE LISTING (82 sequences)

SEQ ID NO: 1 - DNA, 24 bp, Artificial Sequence
  Synthetic Polynucleotide
tcgtcgttttgtcgttttgtcgtt

SEQ ID NO: 2 - DNA, 24 bp, Artificial Sequence
  Synthetic Polynucleotide
ttagggttagggttagggttaggg

SEQ ID NO: 3 - DNA, 24 bp, Artificial Sequence
  Synthetic Polynucleotide
ttattattattattattattatta

SEQ ID NO: 4 - DNA, 24 bp, Artificial Sequence
  Synthetic Polynucleotide
ctagggttagggttagggttaggg

SEQ ID NO: 5 - DNA, 24 bp, Artificial Sequence
  Synthetic Polynucleotide
gtagggttagggttagggttaggg

SEQ ID NO: 6 - DNA, 24 bp, Artificial Sequence
  Synthetic Polynucleotide
atagggttagggttagggttaggg

SEQ ID NO: 7 - DNA, 24 bp, Artificial Sequence
  Synthetic Polynucleotide
tcagggttagggttagggttaggg

SEQ ID NO: 8 - DNA, 24 bp, Artificial Sequence
  Synthetic Polynucleotide
tgagggttagggttagggttaggg

SEQ ID NO: 9 - DNA, 24 bp, Artificial Sequence
  Synthetic Polynucleotide
taagggttagggttagggttaggg

SEQ ID NO: 10 - DNA, 24 bp, Artificial Sequence
  Synthetic Polynucleotide
tttgggttagggttagggttaggg

SEQ ID NO: 11 - DNA, 24 bp, Artificial Sequence
  Synthetic Polynucleotide
ttcgggttagggttagggttaggg

SEQ ID NO: 12 - DNA, 24 bp, Artificial Sequence
  Synthetic Polynucleotide
ttggggttagggttagggttaggg

SEQ ID NO: 13 - DNA, 24 bp, Artificial Sequence
  Synthetic Polynucleotide
ttagggctagggttagggttaggg

SEQ ID NO: 14 - DNA, 24 bp, Artificial Sequence
  Synthetic Polynucleotide
ttaggggtagggttagggttaggg

SEQ ID NO: 15 - DNA, 24 bp, Artificial Sequence
  Synthetic Polynucleotide
ttagggatagggttagggttaggg

SEQ ID NO: 16 - DNA, 24 bp, Artificial Sequence
  Synthetic Polynucleotide
ttagggtcagggttagggttaggg

SEQ ID NO: 17 - DNA, 24 bp, Artificial Sequence
  Synthetic Polynucleotide
ttagggtgagggttagggttaggg

SEQ ID NO: 18 - DNA, 24 bp, Artificial Sequence
  Synthetic Polynucleotide
ttagggtaagggttagggttaggg

SEQ ID NO: 19 - DNA, 24 bp, Artificial Sequence
  Synthetic Polynucleotide
ttagggtttgggttagggttaggg

SEQ ID NO: 20 - DNA, 24 bp, Artificial Sequence
  Synthetic Polynucleotide
ttagggttcgggttagggttaggg

SEQ ID NO: 21 - DNA, 24 bp, Artificial Sequence
  Synthetic Polynucleotide
ttagggttggggttagggttaggg

SEQ ID NO: 22 - DNA, 24 bp, Artificial Sequence
  Synthetic Polynucleotide
ttagggttagggctagggttaggg

SEQ ID NO: 23 - DNA, 24 bp, Artificial Sequence
  Synthetic Polynucleotide
ttagggttaggggtagggttaggg

SEQ ID NO: 24 - DNA, 24 bp, Artificial Sequence
  Synthetic Polynucleotide
ttagggttagggatagggttaggg

SEQ ID NO: 25 - DNA, 24 bp, Artificial Sequence
  Synthetic Polynucleotide
ttagggttagggtcagggttaggg

SEQ ID NO: 26 - DNA, 24 bp, Artificial Sequence
  Synthetic Polynucleotide
ttagggttagggtgagggttaggg

SEQ ID NO: 27 - DNA, 24 bp, Artificial Sequence
  Synthetic Polynucleotide
ttagggttagggtaagggttaggg

SEQ ID NO: 28 - DNA, 24 bp, Artificial Sequence
  Synthetic Polynucleotide
ttagggttagggtttgggttaggg

SEQ ID NO: 29 - DNA, 24 bp, Artificial Sequence
  Synthetic Polynucleotide
ttagggttagggttcgggttaggg

SEQ ID NO: 30 - DNA, 24 bp, Artificial Sequence
  Synthetic Polynucleotide
ttagggttagggttggggttaggg

SEQ ID NO: 31 - DNA, 24 bp, Artificial Sequence
  Synthetic Polynucleotide
ttagggttagggttagggctaggg

SEQ ID NO: 32 - DNA, 24 bp, Artificial Sequence
  Synthetic Polynucleotide
ttagggttagggttaggggtaggg

SEQ ID NO: 33 - DNA, 24 bp, Artificial Sequence
  Synthetic Polynucleotide
ttagggttagggttagggataggg

SEQ ID NO: 34 - DNA, 24 bp, Artificial Sequence
  Synthetic Polynucleotide
ttagggttagggttagggtcaggg

SEQ ID NO: 35 - DNA, 24 bp, Artificial Sequence
  Synthetic Polynucleotide
ttagggttagggttagggtgaggg

SEQ ID NO: 36 - DNA, 24 bp, Artificial Sequence
  Synthetic Polynucleotide
ttagggttagggttagggtaaggg

SEQ ID NO: 37 - DNA, 24 bp, Artificial Sequence
  Synthetic Polynucleotide
ttagggttagggttagggtttggg

SEQ ID NO: 38 - DNA, 24 bp, Artificial Sequence
  Synthetic Polynucleotide
ttagggttagggttagggttcggg

SEQ ID NO: 39 - DNA, 24 bp, Artificial Sequence
  Synthetic Polynucleotide
ttagggttagggttagggttgggg

SEQ ID NO: 40 - DNA, 24 bp, Artificial Sequence
  Synthetic Polynucleotide
ttagggttggggttggggttaggg

SEQ ID NO: 41 - DNA, 18 bp, Artificial Sequence
  Synthetic Polynucleotide
ttagggttagggttaggg

SEQ ID NO: 42 - DNA, 12 bp, Artificial Sequence
  Synthetic Polynucleotide
ttagggttaggg

SEQ ID NO: 43 - DNA, 6 bp, Artificial Sequence
  Synthetic Polynucleotide
ttaggg

SEQ ID NO: 44 - DNA, 18 bp, Artificial Sequence
  Synthetic Polynucleotide
ttattattattattatta

SEQ ID NO: 45 - DNA, 12 bp, Artificial Sequence
  Synthetic Polynucleotide
ttattattatta

SEQ ID NO: 46 - DNA, 6 bp, Artificial Sequence
  Synthetic Polynucleotide
ttatta

SEQ ID NO: 47 - DNA, 24 bp, Artificial Sequence
  Synthetic Polynucleotide
ttaaggttagggttagggttaggg

SEQ ID NO: 48 - DNA, 24 bp, Artificial Sequence
  Synthetic Polynucleotide
ttatggttagggttagggttaggg

SEQ ID NO: 49 - DNA, 24 bp, Artificial Sequence
  Synthetic Polynucleotide
ttacggttagggttagggttaggg

SEQ ID NO: 50 - DNA, 24 bp, Artificial Sequence
  Synthetic Polynucleotide
ttagagttagggttagggttaggg

SEQ ID NO: 51 - DNA, 24 bp, Artificial Sequence
  Synthetic Polynucleotide
ttagtgttagggttagggttaggg

SEQ ID NO: 52 - DNA, 24 bp, Artificial Sequence
  Synthetic Polynucleotide
ttagcgttagggttagggttaggg

SEQ ID NO: 53 - DNA, 24 bp, Artificial Sequence
  Synthetic Polynucleotide
ttaggattagggttagggttaggg

SEQ ID NO: 54 - DNA, 24 bp, Artificial Sequence
  Synthetic Polynucleotide
ttaggtttagggttagggttaggg

SEQ ID NO: 55 - DNA, 24 bp, Artificial Sequence
  Synthetic Polynucleotide
ttaggcttagggttagggttaggg

SEQ ID NO: 56 - DNA, 24 bp, Artificial Sequence
  Synthetic Polynucleotide
ttagggttaaggttagggttaggg

SEQ ID NO: 57 - DNA, 24 bp, Artificial Sequence
  Synthetic Polynucleotide
ttagggttatggttagggttaggg

SEQ ID NO: 58 - DNA, 24 bp, Artificial Sequence
  Synthetic Polynucleotide
ttagggttacggttagggttaggg

SEQ ID NO: 59 - DNA, 24 bp, Artificial Sequence
  Synthetic Polynucleotide
ttagggttagagttagggttaggg

SEQ ID NO: 60 - DNA, 24 bp, Artificial Sequence
  Synthetic Polynucleotide
ttagggttagtgttagggttaggg

SEQ ID NO: 61 - DNA, 24 bp, Artificial Sequence
  Synthetic Polynucleotide
ttagggttagcgttagggttaggg

SEQ ID NO: 62 - DNA, 24 bp, Artificial Sequence
  Synthetic Polynucleotide
ttagggttaggattagggttaggg

SEQ ID NO: 63 - DNA, 24 bp, Artificial Sequence
  Synthetic Polynucleotide
ttagggttaggtttagggttaggg

SEQ ID NO: 64 - DNA, 24 bp, Artificial Sequence
  Synthetic Polynucleotide
ttagggttaggcttagggttaggg

SEQ ID NO: 65 - DNA, 24 bp, Artificial Sequence
  Synthetic Polynucleotide
ttagggttagggttaaggttaggg

SEQ ID NO: 66 - DNA, 24 bp, Artificial Sequence
  Synthetic Polynucleotide
ttagggttagggttatggttaggg

SEQ ID NO: 67 - DNA, 24 bp, Artificial Sequence
  Synthetic Polynucleotide
ttagggttagggttacggttaggg

SEQ ID NO: 68 - DNA, 24 bp, Artificial Sequence
  Synthetic Polynucleotide
ttagggttagggttagagttaggg

SEQ ID NO: 69 - DNA, 24 bp, Artificial Sequence
  Synthetic Polynucleotide
ttagggttagggttagtgttaggg

SEQ ID NO: 70 - DNA, 24 bp, Artificial Sequence
  Synthetic Polynucleotide
ttagggttagggttagcgttaggg

SEQ ID NO: 71 - DNA, 24 bp, Artificial Sequence
  Synthetic Polynucleotide
ttagggttagggttaggattaggg

SEQ ID NO: 72 - DNA, 24 bp, Artificial Sequence
  Synthetic Polynucleotide
ttagggttagggttaggtttaggg

SEQ ID NO: 73 - DNA, 24 bp, Artificial Sequence
  Synthetic Polynucleotide
ttagggttagggttaggcttaggg

SEQ ID NO: 74 - DNA, 24 bp, Artificial Sequence
  Synthetic Polynucleotide
ttagggttagggttagggttaagg

SEQ ID NO: 75 - DNA, 24 bp, Artificial Sequence
  Synthetic Polynucleotide
ttagggttagggttagggttatgg

SEQ ID NO: 76 - DNA, 24 bp, Artificial Sequence
  Synthetic Polynucleotide
ttagggttagggttagggttacgg

SEQ ID NO: 77 - DNA, 24 bp, Artificial Sequence
  Synthetic Polynucleotide
ttagggttagggttagggttagag

SEQ ID NO: 78 - DNA, 24 bp, Artificial Sequence
  Synthetic Polynucleotide
ttagggttagggttagggttagtg

SEQ ID NO: 79 - DNA, 24 bp, Artificial Sequence
  Synthetic Polynucleotide
ttagggttagggttagggttagcg

SEQ ID NO: 80 - DNA, 24 bp, Artificial Sequence
  Synthetic Polynucleotide
ttagggttagggttagggttagga

SEQ ID NO: 81 - DNA, 24 bp, Artificial Sequence
  Synthetic Polynucleotide
ttagggttagggttagggttaggt

SEQ ID NO: 82 - DNA, 24 bp, Artificial Sequence
  Synthetic Polynucleotide
ttagggttagggttagggttaggc